(19) United States
(12) Patent Application Publication
(10) Pub. No.: US 2005/0250121 A1
(43) Pub. Date: Nov. 10, 2005

(54) NTRK2 genetic markers associated with progression of Alzheimer's disease

(75) Inventors: Jeroen Aerssens, Nieuwrode (BE); Maria Athanasiou, Derby, CT (US); Carlos Brain, Somerville, MA (US); Nadine Cohen, Warren, NJ (US); Bradley Dain, Hamden, CT (US); R. Rex Denton, Madison, CT (US); Richard S. Judson, Guilford, CT (US); Vural Ozdemir, Long Beach, CA (US); Carol R. Reed, Bethany, CT (US)

Correspondence Address: STERNE, KESSLER, GOLDSTEIN & FOX PLLC, 1100 NEW YORK AVENUE, N.W., WASHINGTON, DC, 20005, US

(73) Assignee: Genaissance Pharmaceuticals, New Haven, CT (US)

(21) Appl. No.: 10/993,509

(22) Filed: Nov. 22, 2004

Related U.S. Application Data
(60) Provisional application No. 60/524,637, filed on Nov. 24, 2003.

(51) Int. Cl. C12Q 1/68

(52) U.S. Cl. 435/6

(57) ABSTRACT

Haplotypes in the NTRK2 gene associated with progression of Alzheimer's Disease are disclosed. Compositions and methods for detecting these NTRK2 haplotypes are disclosed.

### FIELD OF THE INVENTION

This invention relates to the field of genomics and pharmacogenetics. More specifically, this invention relates to variants of the gene for neurotrophic tyrosine kinase, receptor, type 2 (NTRK2) and their use as predictors of an individual's progression of Alzheimer's Disease (hereinafter, “AD”).

### BACKGROUND OF THE INVENTION

AD is a fatal, progressive, degenerative disorder of the central nervous system. During the course of AD, cognitive, mood, and motor system deficits appear and progressively worsen. In the earliest stages, AD may manifest as Mild Cognitive Impairment (hereinafter, “MCI”), characterized by memory complaints without general cognitive deficits or dementia (Morris et al., Arch. Neurol. 58:397-405 (2001)). Cognitive deficits in AD include difficulty learning and recalling new information, language disorder, disturbances of visuospatial skills and deficits in executive function, all of which increase in severity over the course of the illness. Early in the illness, apathy is apparent and as the illness progresses, agitation becomes increasingly common. In the later stages of the disease, motor system abnormalities manifest (reviewed in Cummings et al., JAMA 287:2335-8 (2002)). AD patients usually survive for 7-10 years after the onset of symptoms (Bracco et al., Arch. Neurol. 51:1213-9 (1994)).

In the United States, the prevalence of AD is estimated at 2.3 million, with a doubling in the prevalence every 5 years after the age of 60 (Brookmeyer et al. Am. J. Public Health 88:1337-42 (1998)). In 1998, the annual cost in the United States for the care of patients with AD was about $40,000 per patient and it is estimated that there will be 14 million AD patients in the United States by the year 2050 (Petersen et al., Neurology 56:1133-42 (2001)). A pharmacological treatment that slows the progression of AD by as little as a year could result in huge cost savings and provide affected individuals with additional time to plan for their future while their decision-making capacity is only minimally affected.

To assess whether a pharmacological treatment is effective in slowing the progression of AD, it is essential to evaluate and detect an alteration in the course of the disease. An evaluation that predicts individuals who are susceptible to a more rapid progression of AD could also be utilized by clinicians to identify patients who may benefit from more aggressive treatment intervention. Furthermore, a method to predict progression of AD may also provide clues to direct the development of new therapeutic agents.

A number of factors have been associated with progression of AD, when considered as the time to institutionalization or the length of survival. Age, gender, marital status (Heyman et al., Neurology 48:1304-9 (1997)), severity of dementia (Heyman et al., supra (1997); Knopman et al., Neurology 52:714-8 (1999)), agitation (Knopman et al., Neurology 52:714-8 (1999)), extrapyramidal signs (Stern et al., Neurology 44:2300-7 (1994)), and higher scores on psychiatric rating scales (Stelle et al., Am. J. Psych. 147:1049-51 (1990) are associated with time to institutionalization. Age (Burns et al., Psychol. Med. 21:363-70 (1991); Heyman et al., Neurology 46:656-60 (1996)), gender (Burns et al., supra; Heyman et al., Neurology 46:656-60 (1996)), age of onset, severity of dementia (Kaszniak et al., Ann. Neurol. 3:246-52 (1978); Diesfeldt et al., Acta. Psychiatr. Scand. 73:366-71 (1986); Burns et al., supra; Heyman et al., supra (1996)), severity of behavioral symptoms (Diesfeldt et al., supra), extrapyramidal signs (Stern et al., supra), and comorbidities (Burns et al., supra) are associated with survival.

In addition to the demographic, symptomatic and comorbid factors associated with AD progression, genetics is thought to play an important role and may account for the large inter-individual variability in disease progression (Farrer et al., Arch. Neurol. 52:918-23 (1995)). Early-onset, dominantly inherited AD may have a more rapid course than late-onset, sporadic AD (Swearer et al., J Geriatr. Psychiatry Neurol. 9:22-5 (1996)). Interestingly, APOE4, an allele that carries an increased risk for developing AD, does not affect disease progression (Corder et al., Neurology 45:1323-8 (1995); Dal Fomo et al., Arch. Neurology 53:345-50 (1996); Koivisto et al., Neuroepidemiology 19:327-32 (2000); Kurz et al., Neurology 47:440-3 (1996)).

A protein that may be involved in the progression of AD is neurotrophic tyrosine kinase receptor type 2 (NTRK2). Also referred to as tyrosine kinase receptor B or TRKB, the gene encoding this protein consists of 24 exons (Stoilov et al., Biochem. Biophys. Res. Commun. 290:1054-65 (2002)) and has been mapped to chromosome 9q22.1 (Nakagawara et al., Genomics 25:538-46 (1995); Valent et al., Europ. J Hum. Genet. 5:102-4 (1997)).

NTRK2 encodes a receptor that binds to brain derived neurotrophic factor, neurotrophin-3, and neurotrophin-4 (Squinto et al., Cell 65:895-903 (1991); Henderson et al., Curr. Opin. Neurobiol. 6:64-70 (1996)). In conditionally gene targeted mice in which knockout of NTRK2 occurs postnatally and is restricted to the forebrain, long term potentiation is impaired at CA1 hippocampal synapses. These mutant mice demonstrate impaired learning behavior or inappropriate coping responses when facing complex and/or stressful learning paradigms. This suggests an essential role for NTRK2 in hippocampus-mediated learning (Minichiello et al. Neuron 24:401-14 (1999)). Since a progressive loss of neurons in the hippocampus and impaired learning behavior are hallmarks of AD (McKhann et al., Neurology 34:939-44 (1984)), NTRK2 may be involved in the progression of AD.

Because of this possible involvement of NTRK2 in progression of AD, it would be useful to assess the degree of variation in the NTRK2 gene in patients with AD and to determine if any variants of this gene are associated with rate of AD progression.

### SUMMARY OF THE INVENTION

Accordingly, the inventors herein have discovered a set of haplotypes in the NTRK2 gene that are associated with the progression of AD. The inventors have also discovered that the copy number of each of these NTRK2 haplotypes affects the progression of AD. The NTRK2 haplotypes are shown in Table 1 below.

TABLE 1
NTRK2 Haplotypes Having Association with
Progression of Alzheimer's Disease

| | Polymorphic Site (PS) | | |
|---|---|---|---|
| Haplotype | 1 | 2 | 3 |
| (1) | | | A |
| (2) | | C | A |
| (3) | C | C | A |

¹The absence of a PS entry for a haplotype indicates that the PS is not part of the marker.

If an individual has (a) two copies of haplotype (1) in Table 1, or (b) one or two copies of any of haplotypes (2)-(3) in Table 1, then that individual is defined as having a “progression marker I” and is more likely to exhibit a slower progression of AD than an individual having (a) one or zero copies of haplotype (1) in Table 1, or (b) zero copies of any of haplotypes (2)-(3) in Table 1, such individual being defined as having a “progression marker II.” Information about the composition of each of haplotypes (1)-(3), namely the location in the NTRK2 gene of each of the polymorphic sites (PSs), and the identity of the reference and variant allele at each PS, can be found in Table 2, shown below.

TABLE 2
Polymorphic Sites Identified in the NTRK2 Gene of
Caucasian Individuals with Alzheimer's Disease

| | | | Position in | | |
|---|---|---|---|---|---|
| PS | | | FIG. 1/ | Reference | Variant |
| Number | Poly ID¹ | Location | SEQ ID NO: 1 | Allele | Allele |
| 1 | 629833643 | intron 1 | 2402 | C | G |
| 2 | 629833648 | intron 2 | 2722 | C | T |
| 3 | 629833652 | intron 2 | 2799 | G | A |

¹The Poly ID is a unique identifier assigned to the indicated PS by Genaissance Pharmaceuticals, Inc., New Haven, CT.

In addition, as described in more detail below, the inventors believe that additional haplotypes may readily be identified based on linkage disequilibrium between any of the above NTRK2 haplotypes and another haplotype located in the NTRK2 gene or another gene, or between an allele at one or more of the PSs in the above haplotypes and an allele at another PS located in the NTRK2 gene or another gene. In particular, such haplotypes include haplotypes that are in linkage disequilibrium with any of haplotypes (1)-(3) in Table 1, hereinafter referred to as “linked haplotypes,” as well as “substitute haplotypes” for any of haplotypes (1)-(3) in Table 1 in which one or more of the polymorphic sites (PSs) in the original haplotype is substituted with another PS, wherein the allele at the substituted PS is in linkage disequilibrium with the allele at the substituting PS.

In one aspect, the invention provides methods and kits for determining whether an individual has a progression marker I or a progression marker II.

In one embodiment, a method is provided for determining whether an individual has a progression marker I or a progression marker II comprising determining whether the individual has (a) two copies, or one or zero copies of any of (i) haplotype (1) in Table 1, (ii) a linked haplotype for haplotype (1) in Table 1, and (iii) a substitute haplotype for haplotype (1) in Table 1, or (b) one or two copies, or zero copies of any of (i) haplotypes (2)-(3) in Table 2, (ii) a linked haplotype for any of haplotypes (2)-(3) in Table 1, and (iii) a substitute haplotype for any of haplotypes (2)-(3) in Table 1.

In another embodiment of the invention, a method is provided for assigning an individual to a first or second progression marker group comprising determining whether the individual has (a) two copies, or one or zero copies of any of (i) haplotype (1) in Table 1, (ii) a linked haplotype for haplotype (1) in Table 1, and (iii) a substitute haplotype for haplotypes (1) in Table 1, or (b) one or two copies, or zero copies of any of (i) haplotypes (2)-(3) in Table 1, (ii) a linked haplotype for any of haplotypes (2)-(3) in Table 1, and (iii) a substitute haplotype for any of haplotypes (2)-(3) in Table 1, and assigning the individual to a progression marker group based on the copy number of that haplotype. The individual is assigned to the first progression marker group if the individual has (a) two copies of any of (i) haplotype (1) in Table 1, (ii) a linked haplotype for haplotype (1) in Table 1, and (iii) a substitute haplotype for haplotype (1) in Table 1, or (b) one or two copies of any of (i) haplotypes (2)-(3) in Table 1, (ii) a linked haplotype for any of haplotypes (2)-(3) in Table 1, and (iii) a substitute haplotype for any of haplotypes (2)-(3) in Table 1, and is assigned to the second progression marker group if the individual has (a) one or zero copies of any of (i) haplotype (1) in Table 1, (ii) a linked haplotype for haplotype (1) in Table 1, and (iii) a substitute haplotype for haplotype (1) in Table 1, or (b) zero copies of any of (i) haplotypes (2)-(3) in Table 1, (ii) a linked haplotype for any of haplotypes (2)-(3) in Table 1, and (iii) a substitute haplotype for any of haplotypes (2)-(3) in Table 1.

One embodiment of a kit for determining whether an individual has a progression marker I or a progression marker II comprises a set of oligonucleotides designed for identifying at least one of the alleles present at each PS in a set of one or more PSs. The set of one or more PSs comprises the set of one or more PSs for any of the haplotypes in Table 1, the set of one or more PSs for a linked haplotype, or the set of one or more PSs for a substitute haplotype. In a further embodiment, the kit comprises a manual with instructions for performing one or more reactions on a human nucleic acid sample to identify the allele(s) present in the individual at each PS in the set and determining if the individual has a progression marker I or a progression marker II based on the identified allele(s).

In yet another embodiment, the invention provides a method for predicting an individual's progression of AD. The method comprises determining whether the individual has a progression marker I or a progression marker II and making a prediction based on the results of the determining step. If the individual is determined to have a progression marker I, then the prediction is that the individual will exhibit a slower progression of AD than an individual not having a progression marker I, and if the individual is determined to have a progression marker II, then the prediction is that the individual will exhibit a faster progression of AD than an individual not having a progression marker II.

### BRIEF DESCRIPTION OF THE FIGURES

FIG. 1A-P illustrates a reference sequence for the NTRK2 gene (contiguous lines; SEQ ID NO:1), with the start and stop positions of each region of coding sequence indicated with a bracket ([or]) and the numerical position below the sequence and the polymorphic site(s) and polymorphism(s) identified by Applicants in the patient cohort indicated by the variant nucleotide positioned below the polymorphic site in the sequence.

### DEFINITIONS

In the context of this disclosure, the terms below shall be defined as follows unless otherwise indicated:

Allele—A particular form of a genetic locus, distinguished from other forms by its particular nucleotide sequence, or one of the alternative polymorphisms found at a polymorphic site.

Gene—A segment of DNA that contains the coding sequence for a protein, wherein the segment may include promoters, exons, introns, and other untranslated regions that control expression.

Genotype—An unphased 5′ to 3′ sequence of nucleotide pair(s) found at a set of one or more polymorphic sites in a locus on a pair of homologous chromosomes in an individual. As used herein, genotype includes a full-genotype and/or a sub-genotype as described below.

Genotyping—A process for determining a genotype of an individual.

Haplotype—A 5′ to 3′ sequence of nucleotides found at a set of one or more polymorphic sites in a locus on a single chromosome from a single individual.

Haplotype pair—The two haplotypes found for a locus in a single individual.

Haplotyping—A process for determining one or more haplotypes in an individual and includes use of family pedigrees, molecular techniques and/or statistical inference.

Haplotype data—Information concerning one or more of the following for a specific gene: a listing of the haplotype pairs in an individual or in each individual in a population; a listing of the different haplotypes in a population; frequency of each haplotype in that or other populations, and any known associations between one or more haplotypes and a trait.

Isolated—As applied to a biological molecule such as RNA, DNA, oligonucleotide, or protein, isolated means the molecule is substantially free of other biological molecules such as nucleic acids, proteins, lipids, carbohydrates, or other material such as cellular debris and growth media. Generally, the term “isolated” is not intended to refer to a complete absence of such material or to absence of water, buffers, or salts, unless they are present in amounts that substantially interfere with the methods of the present invention.

Locus—A location on a chromosome or DNA molecule corresponding to a gene or a physical or phenotypic feature, where physical features include polymorphic sites.

Nucleotide pair—The nucleotides found at a polymorphic site on the two copies of a chromosome from an individual.

Phased—As applied to a sequence of nucleotide pairs for two or more polymorphic sites in a locus, phased means the combination of nucleotides present at those polymorphic sites on a single copy of the locus is known.

Polymorphic site (PS)— A position on a chromosome or DNA molecule at which at least two alternative sequences are found in a population.

Polymorphism—The sequence variation observed in an individual at a polymorphic site. Polymorphisms include nucleotide substitutions, insertions, deletions and microsatellites and may, but need not, result in detectable differences in gene expression or protein function.

Polynucleotide—A nucleic acid molecule comprised of single-stranded RNA or DNA or comprised of complementary, double-stranded DNA.

Population Group—A group of individuals sharing a common ethnogeographic origin.

Reference Population—A group of subjects or individuals who are predicted to be representative of the genetic variation found in the general population. Typically, the reference population represents the genetic variation in the population at a certainty level of at least 85%, preferably at least 90%, more preferably at least 95% and even more preferably at least 99%.

Single Nucleotide Polymorphism (SNP)—Typically, the specific pair of nucleotides observed at a single polymorphic site. In rare cases, three or four nucleotides may be found.

Subject—A human individual whose genotypes or haplotypes or response to treatment or disease state are to be determined.

Treatment—A stimulus administered internally or externally to a subject.

Unphased—As applied to a sequence of nucleotide pairs for two or more polymorphic sites in a locus, unphased means the combination of nucleotides present at those polymorphic sites on a single copy of the locus is not known.

### DESCRIPTION OF THE PREFERRED EMBODIMENTS

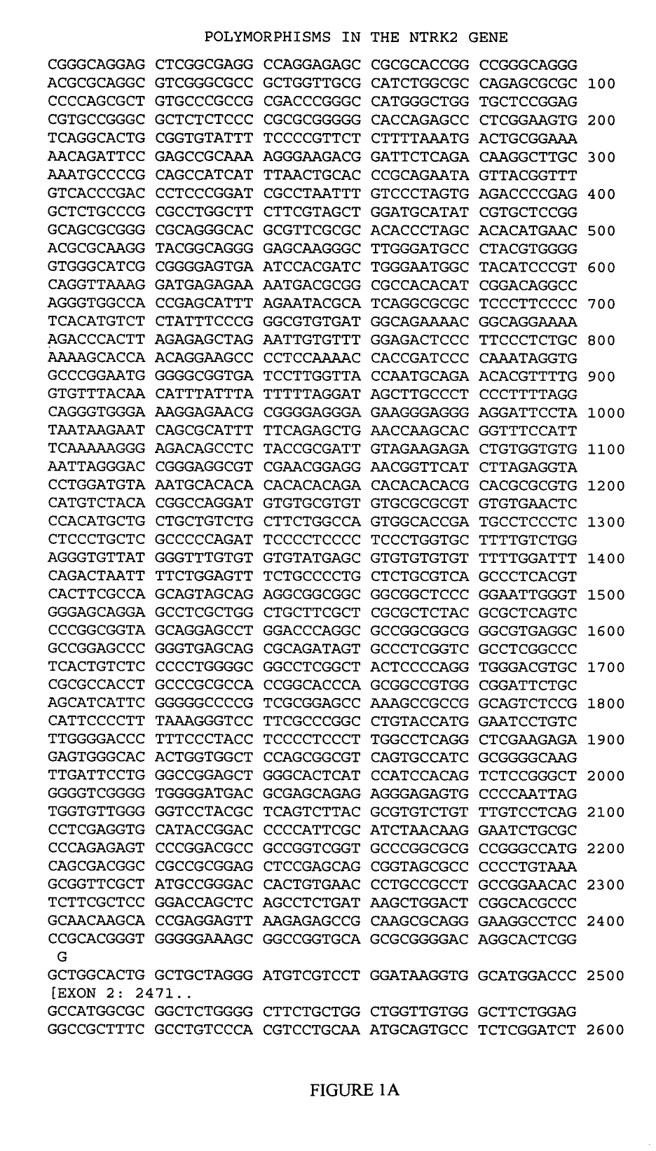

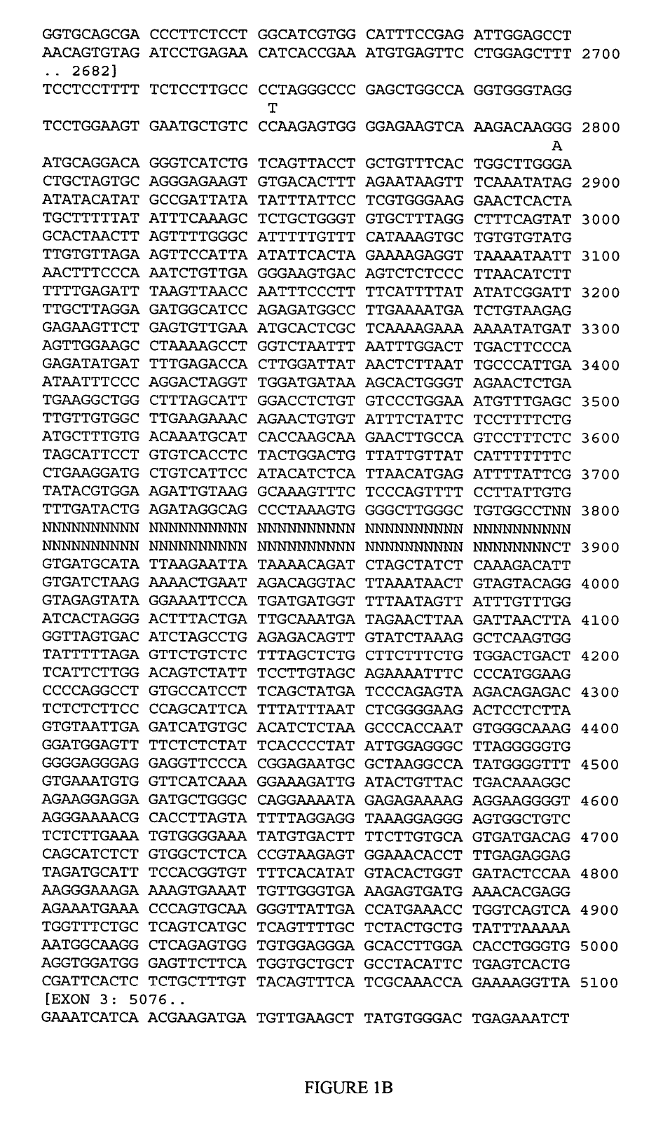

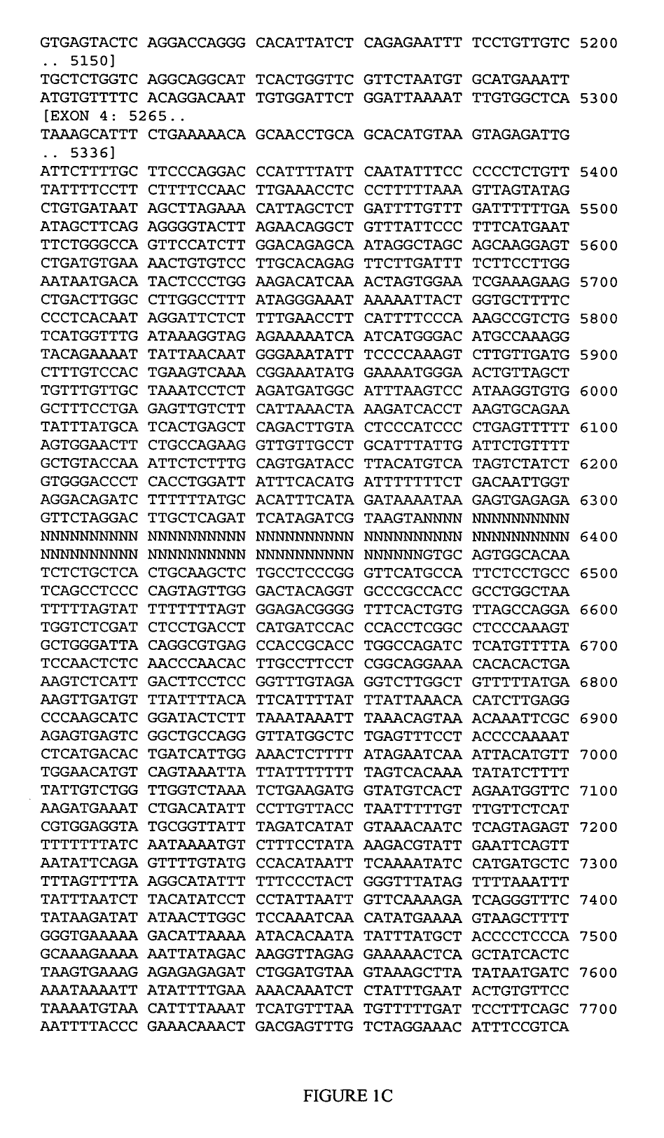

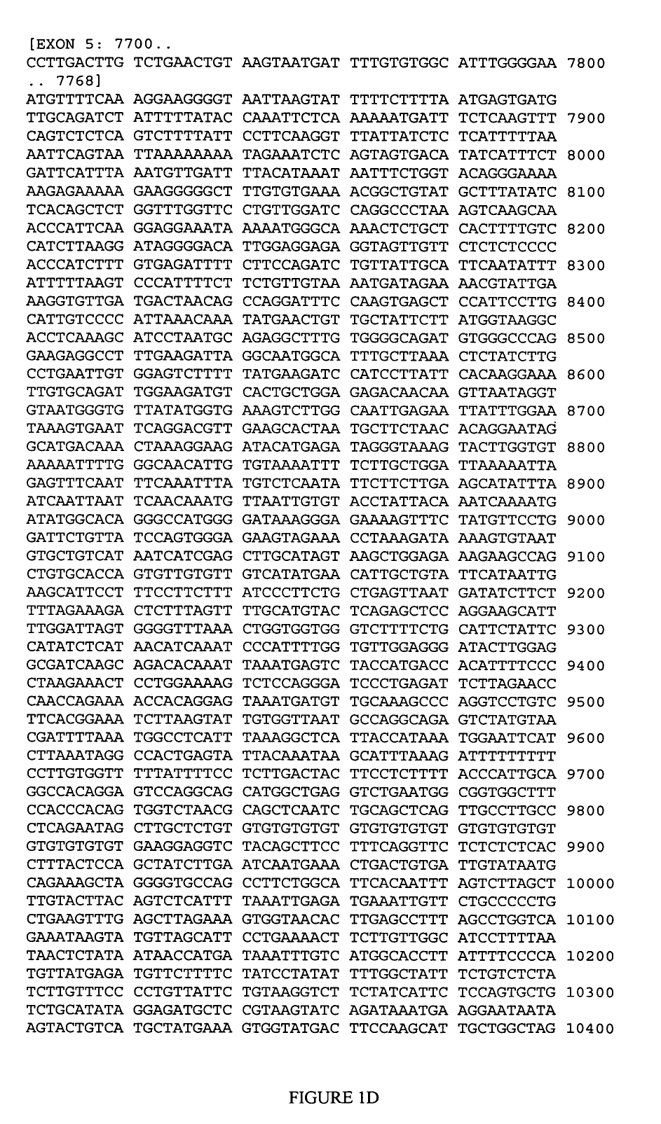

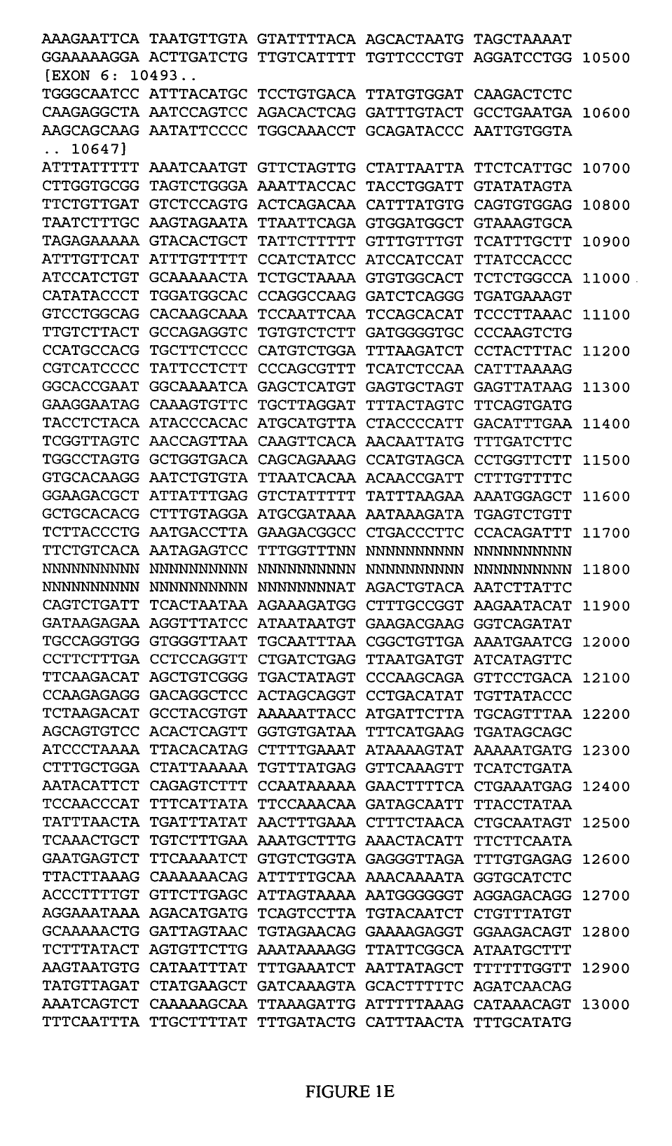

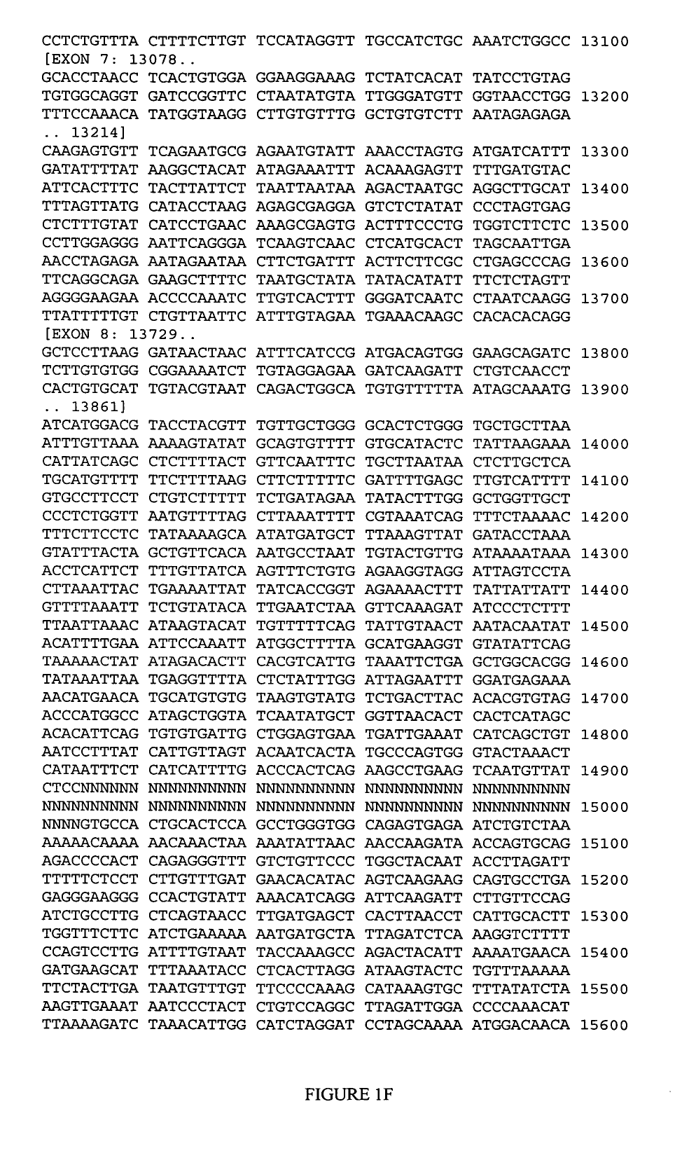

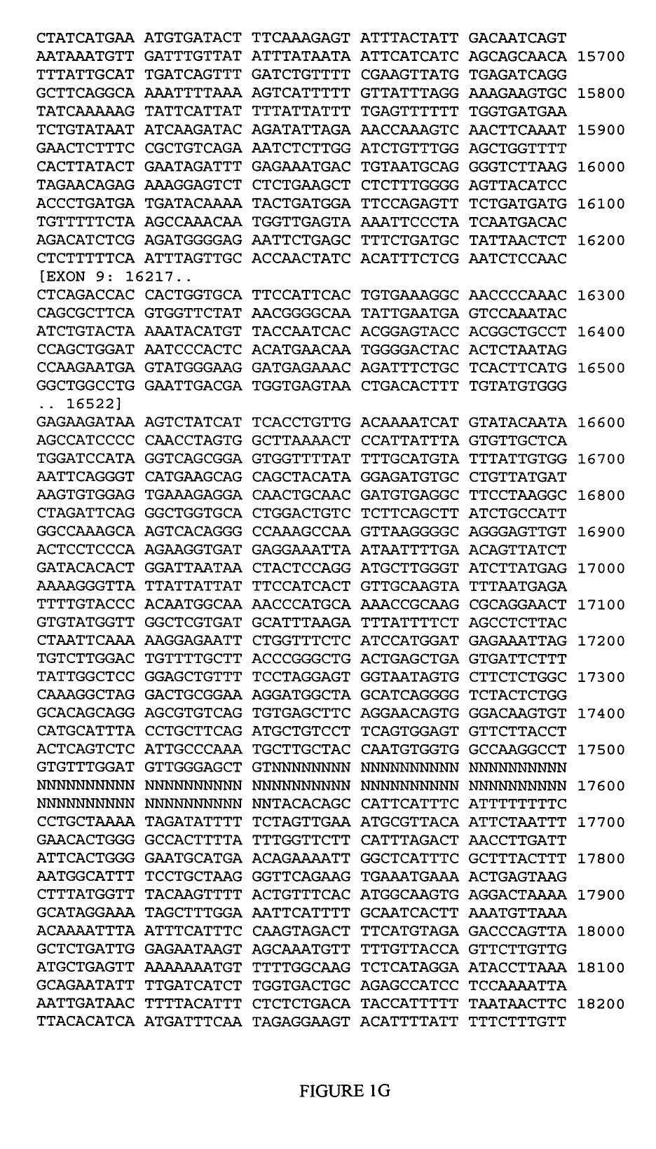

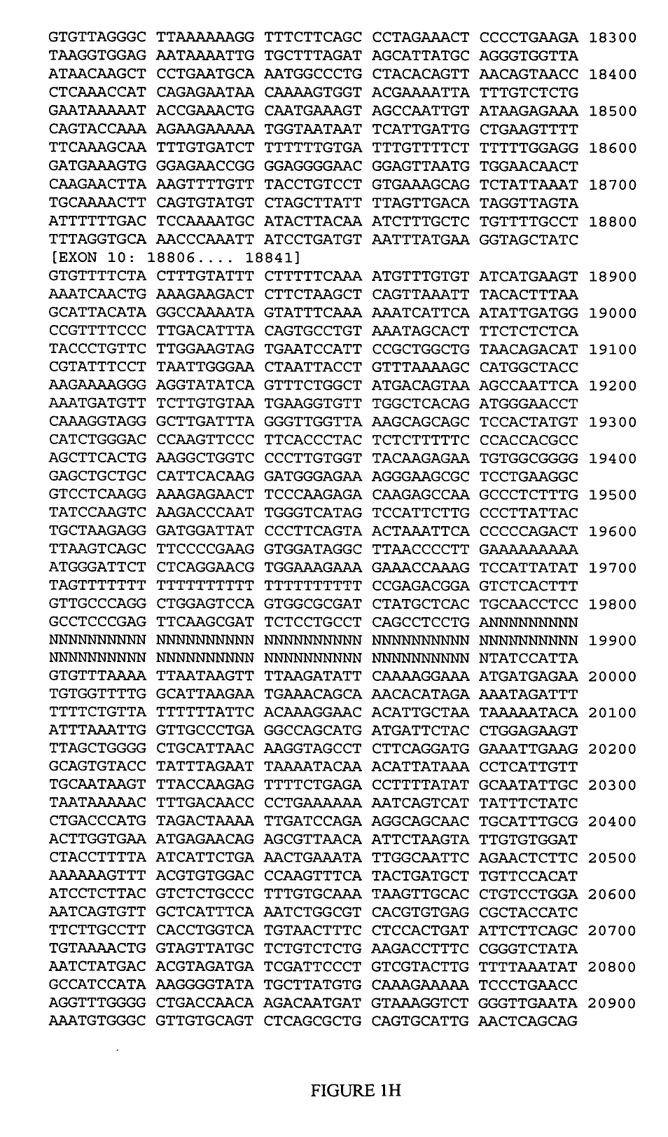

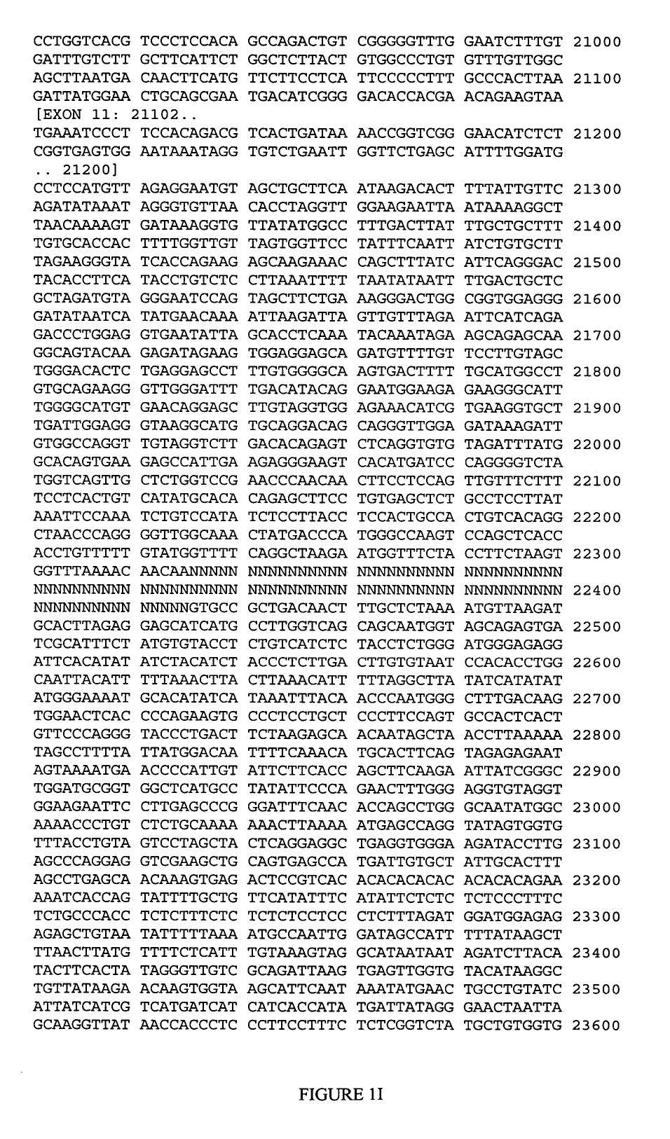

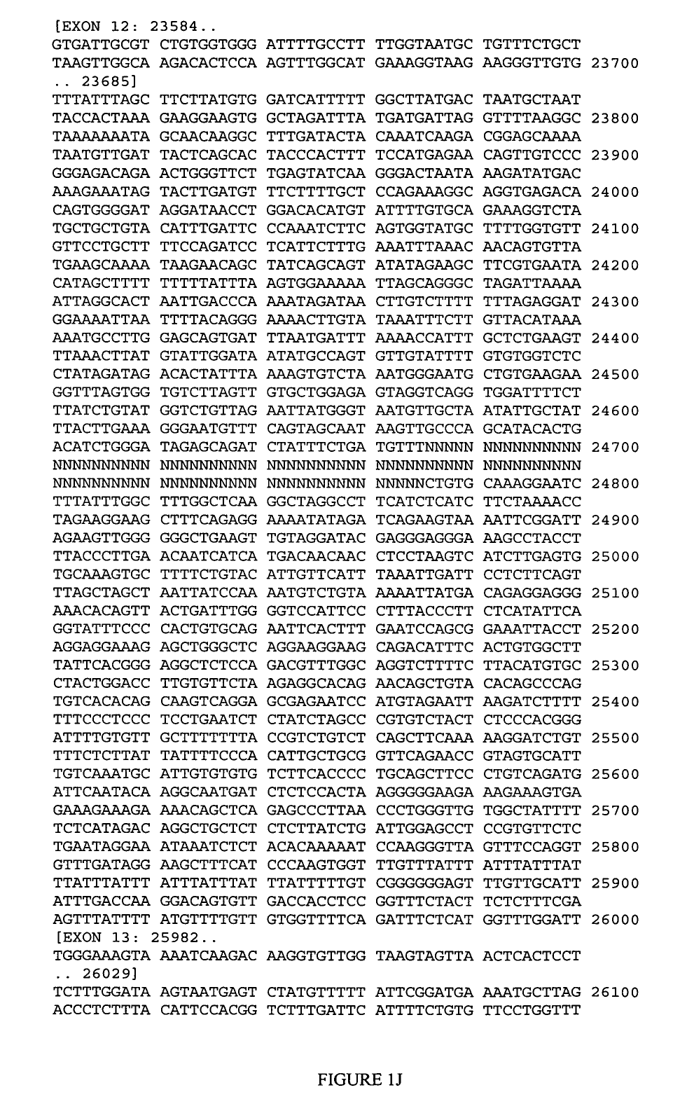

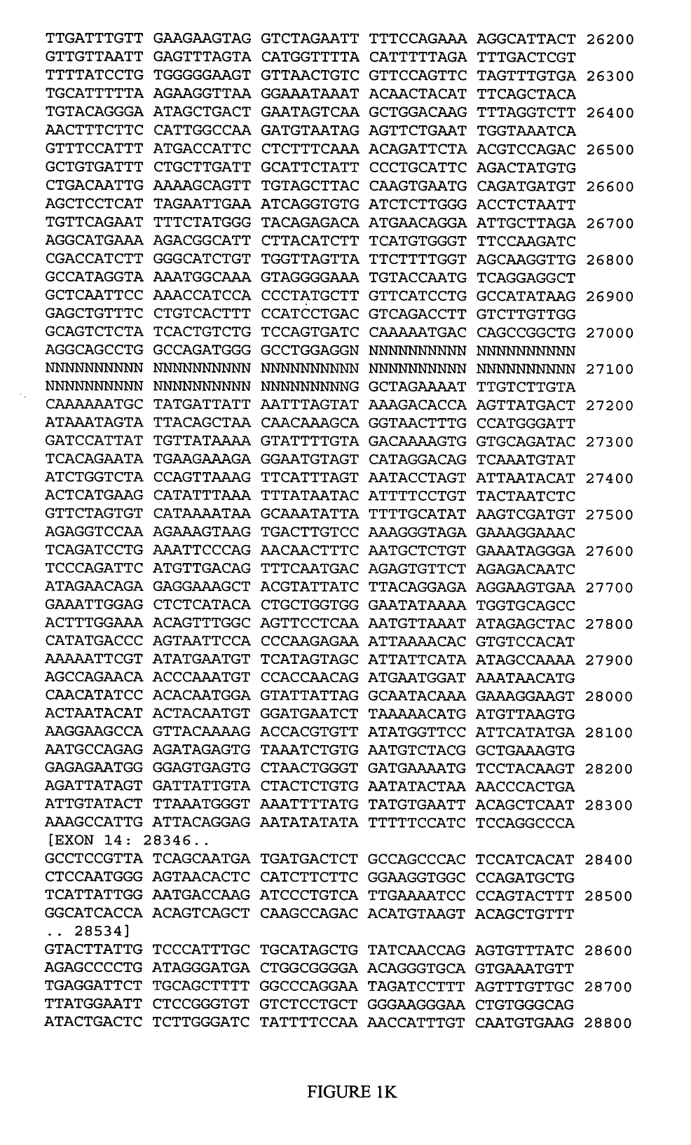

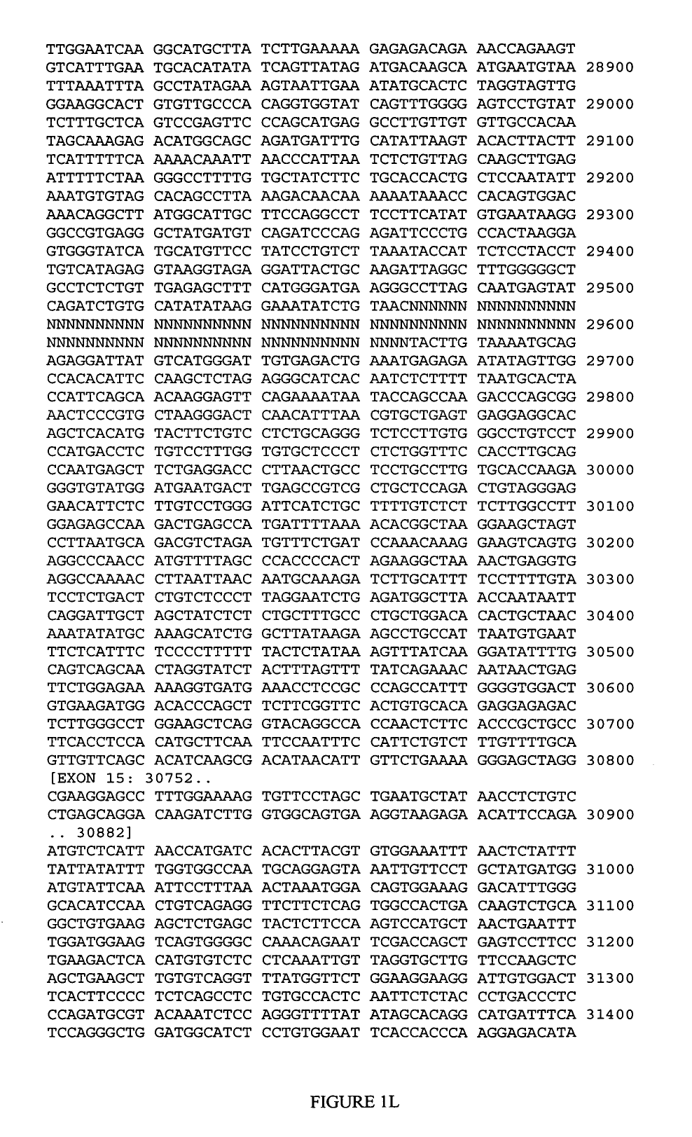

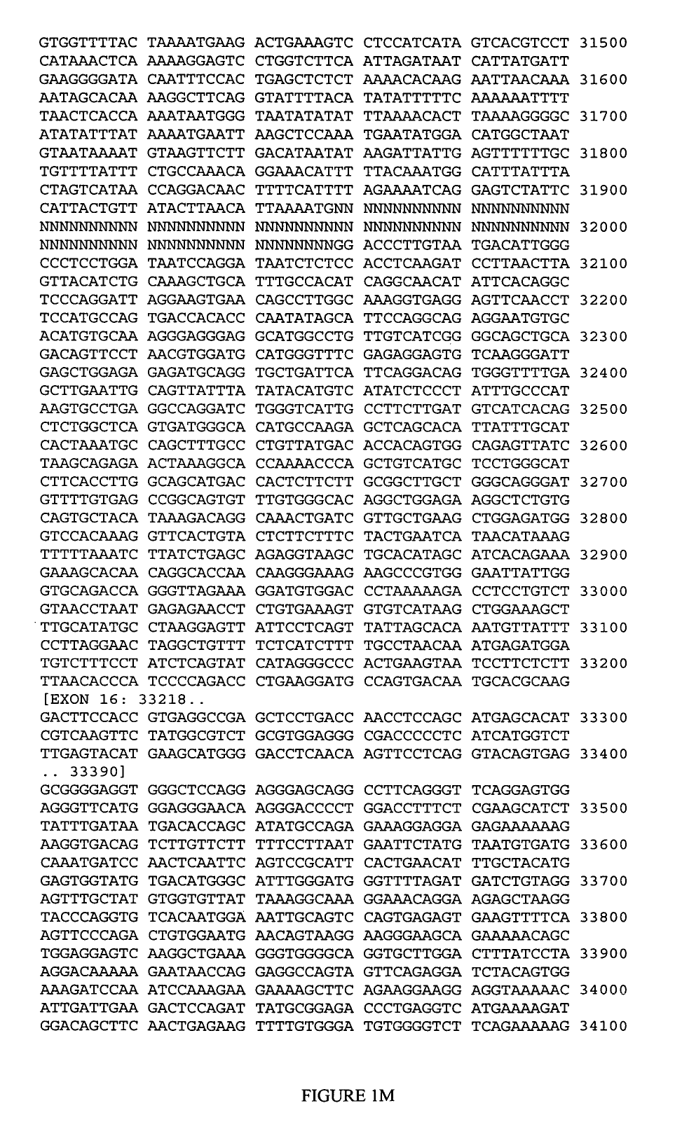

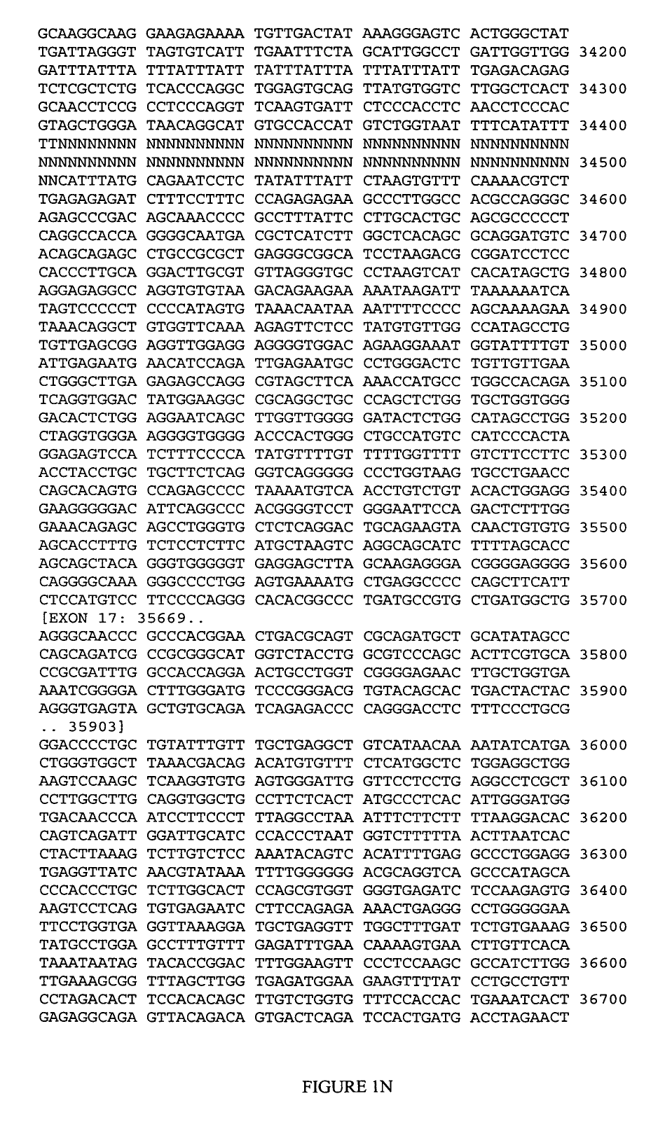

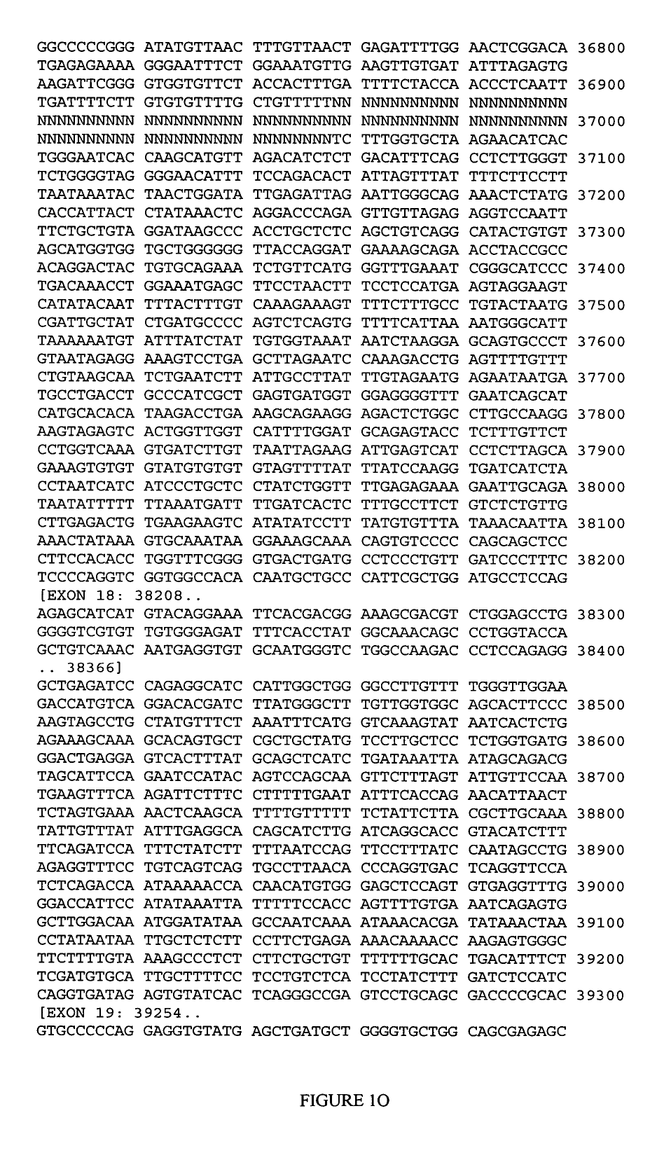

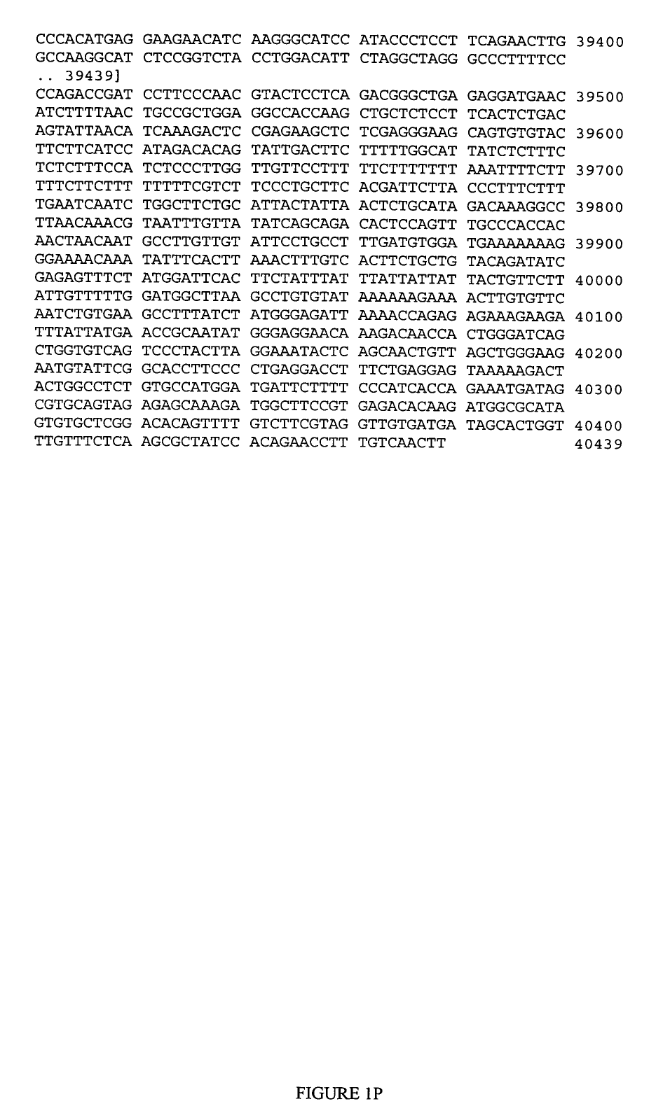

Each disease progression marker of the invention is a combination of a particular haplotype and the copy number for that haplotype. Preferably, the haplotype is one of the haplotypes shown in Table 1. The PS or PSs in these haplotypes are referred to herein as PS1, PS2, and PS3, and are located in the NTRK2 gene at positions corresponding to those identified in FIG. 1/SEQ ID NO:1 (see Table 2 for summary of PS1, PS2, and PS3, and locations). In describing the PSs in the disease progression markers of the invention, reference is made to the sense strand of a gene for convenience. However, as recognized by the skilled artisan, nucleic acid molecules containing a particular gene may be complementary double stranded molecules and thus reference to a particular site or haplotype on the sense strand refers as well to the corresponding site or haplotype on the complementary antisense strand. Further, reference may be made to detecting a genetic marker or haplotype for one strand and it will be understood by the skilled artisan that this includes detection of the complementary haplotype on the other strand.

As described in more detail in the examples below, the disease progression markers of the invention are based on the discovery by the inventors of associations between certain haplotypes in the NTRK2 gene and progression of AD in a cohort of individuals diagnosed with AD.

In particular, the inventors herein discovered that a haplotype comprising adenine at PS3 (haplotype (1) in Table 1) affected the progression of AD of the patients participating in the study. The group of patients having two copies of this haplotype exhibited a slower progression of AD than the patient group having one or zero copies of the haplotype. As used herein, the term “progression” is intended to refer to the rate of decrease in an individual's cognitive function, preferably as measured by the rate of change in his/her scores on the cognitive subscale of the Alzheimer's Disease Assessment (ADAS-cog) (Rosen et al., Am. J. Psychiatry 141:1356-64 (1984); Rockwood et al., J. Neurol. Neurosurg. Psychiatry 71:589-95 (2001); Tariot et al., Neurology 54:2269-76 (2000); Wilcock et al., BMJ 321:1-7 (2000)) administered at two different times. The ADAS-cog measures cognitive function, including spoken language ability, comprehension of spoken language, recall of test instructions, word-finding difficulty in spontaneous speech, following commands, naming objects and fingers, constructional praxis, ideational praxis, orientation, word-recall task and word-recognition task (Alzheimer's Insights Online, Vol. 3, No. 1, 1997). Additionally, an individual's progression of AD may be measured by other scientifically accepted rating scales for cognitive function, including, but not limited to, Behavioral Pathology in Alzheimer's Disease Rating Scale (BEHAVE-AD), Blessed Test, CANTAB (CAmbridge Neuropsychological Test Automated Battery), CERAD (The Consortium to Establish a Registry for Alzheimer's Disease) Clinical and Neuropsychological Tests, Clock Draw Test, Cornell Scale for Depression in Dementia (CSDD), Geriatric Depression Scale (GDS), Mini Mental State Exam (MMSE), Neuropsychiatric Inventory (NPI), and The 7 Minute Screen.

Moreover, as shown in Table 10 below, the different effect of copy number of haplotype (1) on progression of AD is statistically significant. Therefore, this haplotype, in combination with the haplotype copy number, can be used to differentiate the progression of AD that might be observed in an individual having AD. Consequently, two copies of haplotype (1) in Table 1 is referred to herein as a progression marker I, while one or zero copies of haplotype (1) in Table 1 is referred to herein as a progression marker II.

In addition, the skilled artisan would expect that there might be additional PSs in the NTRK2 gene or elsewhere on chromosome 9, wherein an allele at that PS is in high linkage disequilibrium (LD) with an allele at one or more of the PSs in the haplotypes comprising a progression marker I or a progression marker II. Two particular alleles at different PSs are said to be in LD if the presence of the allele at one of the sites tends to predict the presence of the allele at the other site on the same chromosome (Stevens, Mol. Diag. 4:309-17 (1999)). One of the most frequently used measures of linkage disequilibrium is Δ², which is calculated using the formula described by Devlin et al. (Genomics 29(2):311-22 (1995)). Δ² is the measure of how well an allele X at a first PS predicts the occurrence of an allele Y at a second PS on the same chromosome. The measure only reaches 1.0 when the prediction is perfect (e.g., X if and only if Y).

Thus, the skilled artisan would expect that all of the embodiments of the invention described herein may frequently be practiced by substituting any (or all) of the specifically identified NTRK2 PSs in a progression marker with another PS, wherein an allele at the substituted PS is in LD with an allele at the “substituting” PS. This “substituting” PS may be one that is currently known or subsequently discovered and may be present in the NTRK2 gene, in a genomic region of about 100 kilobases spanning the NTRK2 gene, or elsewhere on chromosome 9.

Further, the inventors contemplate that there will be other haplotypes in the NTRK2 gene or elsewhere on chromosome 9 that are in LD with one or more of the haplotypes in Table 1 that would therefore also be predictive of progression of AD. Preferably, the linked haplotype is present in the NTRK2 gene or in a genomic region of about 100 kilobases spanning the NTRK2 gene. The linkage disequilibrium between the haplotypes in Table 1 and such linked haplotypes can also be measured using Δ².

In preferred embodiments, the linkage disequilibrium between an allele at a polymorphic site in any of the haplotypes in Table 1 and an allele at a “substituting” polymorphic site, or between any of the haplotypes in Table 1 and a linked haplotype, has a Δ² value, as measured in a suitable reference population, of at least 0.75, more preferably at least 0.80, even more preferably at least 0.85 or at least 0.90, yet more preferably at least 0.95, and most preferably 1.0. A suitable reference population for this Δ² measurement is preferably a population for which the distribution of its members reflects that of the population of patients having AD. The reference population may be the general population, a population having AD or AD risk factors, or the like.

LD patterns in genomic regions are readily determined empirically in appropriately chosen samples using various techniques known in the art for determining whether any two alleles (either those occurring at two different PSs or two haplotypes for two different multi-site loci) are in linkage disequilibrium (GENETIC DATA ANALYSIS II, Weir, Sinauer Associates, Inc. Publishers, Sunderland, MA, 1996). The skilled artisan may readily select which method of determining LD will be best suited for a particular sample size and genomic region.

As described above and in the examples below, the progression markers of the invention are associated with changes in the cognitive subscale of the Alzheimer's Disease Assessment Scale (ADAS-cog) administered at two different times. Thus, the invention provides a method and kit for determining whether an individual has a progression marker I or a progression marker II. A progression marker I is (a) two copies of any of (i) haplotype (1) in Table 1, (ii) a linked haplotype for haplotype (1) in Table 1, and (iii) a substitute haplotype for haplotype (1) in Table 1, or (b) one or two copies of any of (i) haplotypes (2)-(3) in Table 2, (ii) a linked haplotype for any of haplotypes (2)-(3) in Table 1, and (iii) a substitute haplotype for any of haplotypes (2)-(3) in Table 1. A progression marker II is (a) one or zero copies of any of (i) haplotype (1) in Table 1, (ii) a linked haplotype for haplotype (1) in Table 1, and (iii) a substitute haplotype for haplotype (1) in Table 1, or (b) zero copies of any of (i) haplotypes (2)-(3) in Table 2, (ii) a linked haplotype for any of haplotypes (2)-(3) in Table 1, and (iii) a substitute haplotype for any of haplotypes (2)-(3) in Table 1.

In one embodiment, the invention provides a method for determining whether an individual has a progression marker I or a progression marker II. The method comprises determining whether the individual has (a) two copies, or one or zero copies of any of (i) haplotype (1) in Table 1, (ii) a linked haplotype for haplotype (1) in Table 1, and (iii) a substitute haplotype for haplotype (1) in Table 1, or (b) one or two copies, or zero copies of any of (i) haplotypes (2)-(3) in Table 2, (ii) a linked haplotype for any of haplotypes (2)-(3) in Table 1, and (iii) a substitute haplotype for any of haplotypes (2)-(3) in Table 1.

In some embodiments, the individual is Caucasian and may be diagnosed with a cognitive disorder, such as mild to moderate dementia of the Alzheimer's type, dementia associated with Parkinson's Disease, MCI, a vascular dementia, and Lewy body dementia, or may have risk factors associated with a cognitive disorder.

In another embodiment, the invention provides a method for assigning an individual to a first or second progression marker group. The method comprises determining whether the individual has (a) two copies, or one or zero copies of any of (i) haplotype (1) in Table 1, (ii) a linked haplotype for haplotype (1) in Table 1, and (iii) a substitute haplotype for haplotype (1) in Table 1, or (b) one or two copies, or zero copies of any of (i) haplotypes (2)-(3) in Table 2, (ii) a linked haplotype for any of haplotypes (2)-(3) in Table 1, and (iii) a substitute haplotype for any of haplotypes (2)-(3) in Table 1, and assigning the individual to the first progression marker group if the individual has (a) two copies of any of (i) haplotype (1) in Table 1, (ii) a linked haplotype for haplotype (1) in Table 1, and (iii) a substitute haplotype for haplotype (1) in Table 1, or (b) one or two copies of any of (i) haplotypes (2)-(3) in Table 2, (ii) a linked haplotype for any of haplotypes (2)-(3) in Table 1, and (iii) a substitute haplotype for any of haplotypes (2)-(3) in Table 1, and assigning the individual to the second progression marker group if the individual has (a) one or zero copies of any of (i) haplotype (1) in Table 1, (ii) a linked haplotype for haplotype (1) in Table 1, and (iii) a substitute haplotype for haplotype (1) in Table 1, or (b) zero copies of any of (i) haplotypes (2)-(3) in Table 2, (ii) a linked haplotype for any of haplotypes (2)-(3) in Table 1, and (iii) a substitute haplotype for any of haplotypes (2)-(3) in Table 1.

In some embodiments, the individual is Caucasian and may be diagnosed with a cognitive disorder, such as mild to moderate dementia of the Alzheimer's type, dementia associated with Parkinson's Disease, MCI, a vascular dementia, and Lewy body dementia, or may have risk factors associated with a cognitive disorder.

The presence in an individual of a progression marker I or a progression marker II may be determined by a variety of indirect or direct methods well known in the art for determining haplotypes or haplotype pairs for a set of one or more PSs in one or both copies of the individual's genome, including those discussed below. The genotype for a PS in an individual may be determined by methods known in the art or as described below.

One indirect method for determining whether zero copies, one copy, or two copies of a haplotype is present in an individual is by prediction based on the individual's genotype determined at one or more of the PSs comprising the haplotype and using the determined genotype at each site to determine the haplotypes present in the individual. The presence of zero copies, one copy, or two copies of a haplotype of interest can be determined by visual inspection of the alleles at the PS that comprise the haplotype. The haplotype pair is assigned by comparing the individual's genotype with the genotypes at the same set of PS corresponding to the haplotype pairs known to exist in the general population or in a specific population group or to the haplotype pairs that are theoretically possible based on the alternative alleles possible at each PS, and determining which haplotype pair is most likely to exist in the individual.

In a related indirect haplotyping method, the presence in an individual of zero copies, one copy, or two copies of a haplotype is predicted from the individual's genotype for a set of PSs comprising the selected haplotype using information on haplotype pairs known to exist in a reference population. In one embodiment, this haplotype pair prediction method comprises identifying a genotype for the individual at the set of PSs comprising the selected haplotype, accessing data containing haplotype pairs identified in a reference population for a set of PSs comprising the PSs of the selected haplotype, and assigning to the individual a haplotype pair that is consistent with the individual's genotype. Whether the individual has a disease progression marker I or a disease progression marker II can be subsequently determined based on the assigned haplotype pair. The haplotype pair can be assigned by comparing the individual's genotype with the genotypes corresponding to the haplotype pairs known to exist in the general population or in a specific population group, and determining which haplotype pair is consistent with the genotype of the individual. In some embodiments, the comparing step may be performed by visual inspection. When the genotype of the individual is consistent with more than one haplotype pair, frequency data may be used to determine which of these haplotype pairs is most likely to be present in the individual. If a particular haplotype pair consistent with the genotype of the individual is more frequent in the reference population than other pairs consistent with the genotype, then that haplotype pair with the highest frequency is the most likely to be present in the individual. The haplotype pair frequency data used in this determination is preferably for a reference population coimprising the same ethnogeographic group as the individual. This determination may also be performed in some embodiments by visual inspection. In other embodiments, the comparison may be made by a computer-implemented algorithm with the genotype of the individual and the reference haplotype data stored in computer-readable formats. For example, as described in WO 01/80156, one computer-implemented algorithm to perform this comparison entails enumerating all possible haplotype pairs which are consistent with the genotype, accessing data containing haplotype pairs frequency data determined in a reference population to determine a probability that the individual has a possible haplotype pair, and analyzing the determined probabilities to assign a haplotype pair to the individual.

Typically, the reference population is composed of randomly selected individuals representing the major ethnogeographic groups of the world. A preferred reference population for use in the methods of the present invention consists of Caucasian individuals, the number of which is chosen based on how rare a haplotype is that one wants to be guaranteed to see. For example, if one wants to have a q % chance of not missing a haplotype that exists in the population at a p % frequency of occurring in the reference population, the number of individuals (n) who must be sampled is given by 2n=log(1−q)/log(1−p) where p and q are expressed as fractions. A preferred reference population allows the detection of any haplotype whose frequency is at least 10% with about 99% certainty. A particularly preferred reference population includes a 3-generation Caucasian family to serve as a control for checking quality of haplotyping procedures.

If the reference population comprises more than one ethnogeographic group, the frequency data for each group is examined to determine whether it is consistent with Hardy-Weinberg equilibrium. Hardy-Weinberg equilibrium (PRINCIPLES OF POPULATION GENOMICS, 3^{rd} ed., Hartl, Sinauer Associates, Sunderland, Mass., 1997) postulates that the frequency of finding the haplotype pair H₁/H₂ is equal to P_{H-W}(H₁/H₂)=2p(H₁)p(H₂) if H₁≠H₂ and p_{H-W}(H₁/H₂)=p(H₁)p(H₂) if H₁=H₂. A statistically significant difference between the observed and expected haplotype frequencies could be due to one or more factors including significant inbreeding in the population group, strong selective pressure on the gene, sampling bias, and/or errors in the genotyping process. If large deviations from Hardy-Weinberg equilibrium are observed in an ethnogeographic group, the number of individuals in that group can be increased to see if the deviation is due to a sampling bias. If a larger sample size does not reduce the difference between observed and expected haplotype pair frequencies, then one may wish to consider haplotyping the individual using a direct haplotyping method such as, for example, CLASPER System™ technology ((U.S. Pat. No. 5,866,404), single molecule dilution, or allele-specific long-range PCR (Michalotos-Beloin et al., Nucleic Acids Res. 24:4841-3 (1996)).

In one embodiment of this method for predicting a haplotype pair for an individual, the assigning step involves performing the following analysis. First, each of the possible haplotype pairs is compared to the haplotype pairs in the reference population. Generally, only one of the haplotype pairs in the reference population matches a possible haplotype pair and that pair is assigned to the individual. Occasionally, only one haplotype represented in the reference haplotype pairs is consistent with a possible haplotype pair for an individual, and in such cases the individual is assigned a haplotype pair containing this known haplotype and a new haplotype derived by subtracting the known haplotype from the possible haplotype pair. Alternatively, the haplotype pair in an individual may be predicted from the individual's genotype for that gene using reported methods (e.g., Clark et al., Mol. Biol. Evol. 7:111-22 (1990) or WO 01/80156) or through a commercial haplotyping service such as offered by Genaissance Pharmaceuticals, Inc. (New Haven, Conn.). In rare cases, either no haplotypes in the reference population are consistent with the possible haplotype pairs, or alternatively, multiple reference haplotype pairs are consistent with the possible haplotype pairs. In such cases, the individual is preferably haplotyped using a direct molecular haplotyping method such as, for example, CLASPER System™ technology (U.S. Pat. No. 5,866,404), SMD, or allele-specific long-range PCR (Michalotos-Beloin et al., supra).

Determination of the number of haplotypes present in the individual from the genotypes is illustrated here for haplotype (1) in Table 1. Table 3 below shows the 3 (3ⁿ, where each of n bi-allelic polymorphic sites may have one of 3 different genotypes present) genotypes that may be detected at PS3, using both chromosomal copies from an individual. Each of the possible genotypes for this site allow unambiguous determination of the number of copies of the haplotype (1) in Table 1 present in the individual. For instances where there is ambiguity in the haplotype pair underlying the determined genotype (i.e., when two or more PSs are included in the haplotype), frequency information may be used to determine the most probable haplotype pair and therefore the most likely number of copies of the haplotype in the individual. If a particular haplotype pair consistent with the genotype of the individual is more frequent in the reference population than other pairs consistent with the genotype, then that haplotype pair with the highest frequency is the most likely to be present in the individual. The copy number of the haplotype of interest in this haplotype pair can then be determined by visual inspection of the alleles at the PS that comprise the response marker for each haplotype in the pair.

Although this illustration has been directed to the particular instance of determining the number of copies of haplotype (1) in Table 1 present in an individual, the process would be analogous for the other haplotypes shown in Table 1, or for the linked haplotypes or substitute haplotypes for any of the haplotypes in Table 1.

TABLE 3
Possible Copy Numbers of Haplotype (1)
in Table 1 Based on Genotypes at PS1

| | Copy Number of |
|---|---|
| | Haploytpe (1) in |
| PS3 | Table 1 |
| G/G | 2 |
| G/A | 1 |
| A/A | 0 |

The individual's genotype for the desired set of PS may be determined using a variety of methods well-known in the art. Such methods typically include isolating from the individual a genomic DNA sample comprising both copies of the gene or locus of interest, amplifying from the sample one or more target regions containing the polymorphic sites to be genotyped, and detecting the nucleotide pair present at each PS of interest in the amplified target region(s). It is not necessary to use the same procedure to determine the genotype for each PS of interest.

In addition, the identity of the allele(s) present at any of the novel PSs described herein may be indirectly determined by haplotyping or genotyping another PS having an allele that is in linkage disequilibrium with an allele of the PS that is of interest. PSs having an allele in linkage disequilibrium with an allele of the presently disclosed PSs may be located in regions of the gene or in other genomic regions not examined herein. Detection of the allele(s) present at a PS, wherein the allele is in linkage disequilibrium with an allele of the novel PSs described herein may be performed by, but is not limited to, any of the above-mentioned methods for detecting the identity of the allele at a PS.

Alternatively, the presence in an individual of a haplotype or haplotype pair for a set of PSs comprising a response marker may be determined by directly haplotyping at least one of the copies of the individual's genomic region of interest, or suitable fragment thereof, using methods known in the art. Such direct haplotyping methods typically involve treating a genomic nucleic acid sample isolated from the individual in a manner that produces a hemizygous DNA sample that only has one of the two “copies” of the individual's genomic region which, as readily understood by the skilled artisan, may be the same allele or different alleles, amplifying from the sample one or more target regions containing the PSs to be genotyped, and detecting the nucleotide present at each PS of interest in the amplified target region(s). The nucleic acid sample may be obtained using a variety of methods known in the art for preparing hemizygous DNA samples, which include: targeted in vivo cloning (TIVC) in yeast as described in WO 98/01573, U.S. Pat. No. 5,866,404, and U.S. Pat. No. 5,972,614; generating hemizygous DNA targets using an allele specific oligonucleotide in combination with primer extension and exonuclease degradation as described in U.S. Pat. No. 5,972,614; single molecule dilution (SMD) as described in Ruaño et al., Proc. Natl. Acad. Sci. 87:6296-300 (1990); and allele specific PCR (Ruaño et al., Nucl. Acids Res. 17:8392 (1989); Ruaño et al., Nucl. Acids Res. 19:6877-82 (1991); Michalatos-Beloin et al., supra).

As will be readily appreciated by those skilled in the art, any individual clone will typically only provide haplotype information on one of the two genomic copies present in an individual. If haplotype information is desired for the individual's other copy, additional clones will usually need to be examined. Typically, at least five clones should be examined to have more than a 90% probability of haplotyping both copies of the genomic locus in an individual. In some cases, however, once the haplotype for one genomic allele is directly determined, the haplotype for the other allele may be inferred if the individual has a known genotype for the PSs of interest or if the haplotype frequency or haplotype pair frequency for the individual's population group is known.

While direct haplotyping of both copies of the gene is preferably performed with each copy of the gene being placed in separate containers, it is also envisioned that direct haplotyping could be performed in the same container if the two copies are labeled with different tags, or are otherwise separately distinguishable or identifiable. For example, if first and second copies of the gene are labeled with different first and second fluorescent dyes, respectively, and an allele-specific oligonucleotide labeled with yet a third different fluorescent dye is used to assay the PS(s), then detecting a combination of the first and third dyes would identify the polymorphism in the first gene copy while detecting a combination of the second and third dyes would identify the polymorphism in the second gene copy.

The nucleic acid sample used in the above indirect and direct haplotyping methods is typically isolated from a biological sample taken from the individual, such as a blood sample or tissue sample. Suitable tissue samples include whole blood, saliva, tears, urine, skin and hair.

The target region(s) containing the PS of interest may be amplified using any oligonucleotide-directed amplification method, including but not limited to polymerase chain reaction (PCR) (U.S. Pat. No. 4,965,188), ligase chain reaction (LCR) (Barany et al., Proc. Natl. Acad. Sci. USA 88:189-93 (1991); WO 90/01069), and oligonucleotide ligation assay (OLA) (Landegren et al., Science 241:1077-80 (1988)). Other known nucleic acid amplification procedures may be used to amplify the target region(s) including transcription-based amplification systems (U.S. Pat. No. 5,130,238; European Patent No. EP 329,822; U.S. Pat. No. 5,169,766; WO 89/06700) and isothermal methods (Walker et al., Proc. Natl. Acad. Sci. USA 89:392-6 (1992)).

In both the direct and indirect haplotyping methods, the identity of a nucleotide (or nucleotide pair) at a PS(s) in the amplified target region may be determined by sequencing the amplified region(s) using conventional methods. If both copies of the gene are represented in the amplified target, it will be readily appreciated by the skilled artisan that only one nucleotide will be detected at a PS in individuals who are homozygous at that site, while two different nucleotides will be detected if the individual is heterozygous for that site. The polymorphism may be identified directly, known as positive-type identification, or by inference, referred to as negative-type identification. For example, where a polymorphism is known to be guanine and cytosine in a reference population, a site may be positively determined to be either guanine or cytosine for an individual homozygous at that site, or both guanine and cytosine, if the individual is heterozygous at that site. Alternatively, the site may be negatively determined to be not guanine (and thus cytosine/cytosine) or not cytosine (and thus guanine/guanine).

A PS in the target region may also be assayed before or after amplification using one of several hybridization-based methods known in the art. Typically, allele-specific oligonucleotides are utilized in performing such methods. The allele-specific oligonucleotides may be used as differently labeled probe pairs, with one member of the pair showing a perfect match to one variant of a target sequence and the other member showing a perfect match to a different variant. In some embodiments, more than one PS may be detected at once using a set of allele-specific oligonucleotides or oligonucleotide pairs. Preferably, the members of the set have melting temperatures within 5° C., and more preferably within 2° C., of each other when hybridizing to each of the polymorphic sites being detected.

Hybridization of an allele-specific oligonucleotide to a target polynucleotide may be performed with both entities in solution, or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking baking, etc. Allele-specific oligonucleotides may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibers, chips, dishes, and beads. The solid support may be treated, coated or derivatized to facilitate the immobilization of the allele-specific oligonucleotide or target nucleic acid.

Detecting the nucleotide or nucleotide pair at a PS of interest may also be determined using a mismatch detection technique, including but not limited to the RNase protection method using riboprobes (Winter et al., Proc. Natl. Acad. Sci. USA 82:7575 (1985); Meyers et al., Science 230:1242 (1985)) and proteins which recognize nucleotide mismatches, such as the E. coli mutS protein (Modrich, Ann. Rev. Genet. 25:229-53 (1991)). Alternatively, variant alleles can be identified by single strand conformation polymorphism (SSCP) analysis (Orita et al., Genomics 5:874-9 (1989); Humphries et al., in MOLECULAR DIAGNOSIS OF GENETIC DISEASES, Elles, ed., pp. 321-340, 1996) or denaturing gradient gel electrophoresis (DGGE) (Wartell et al., Nucl. Acids Res. 18:2699-706 (1990); Sheffield et al., Proc. Natl. Acad. Sci. USA 86:232-6 (1989)).

A polymerase-mediated primer extension method may also be used to identify the polymorphism(s). Several such methods have been described in the patent and scientific literature and include the “Genetic Bit Analysis” method (WO 92/15712) and the ligase/polymerase mediated genetic bit analysis (U.S. Pat. No. 5,679,524. Related methods are disclosed in WO 91/02087, WO 90/09455, WO 95/17676, and U.S. Pat. Nos. 5,302,509 and 5,945,283. Extended primers containing the complement of the polymorphism may be detected by mass spectrometry as described in U.S. Pat. No. 5,605,798. Another primer extension method is allele-specific PCR (Ruaño et al., 1989, supra; Ruaño et al., 1991, supra; WO 93/22456; Turki et al., J. Clin. Invest. 95:1635-41 (1995)). In addition, multiple PSs may be investigated by simultaneously amplifying multiple regions of the nucleic acid using sets of allele-specific primers as described in WO 89/10414.

The genotype or haplotype for the NTRK2 gene of an individual may also be determined by hybridization of a nucleic acid sample containing one or both copies of the gene, mRNA, cDNA or fragment(s) thereof, to nucleic acid arrays and subarrays such as described in WO 95/11995. The arrays would contain a battery of allele-specific oligonucleotides representing each of the PSs to be included in the genotype or haplotype.

The invention also provides a kit for determining whether an individual has a progression marker I or a progression marker II. The kit comprises a set of one or more oligonucleotides designed for identifying at least one of the alleles at each PS in a set of one or more PSs, wherein the set of one or more PSs comprises (a) PS3; (b) PS2 and PS3; (c) PS1, PS2, and PS3; (d) a set of one or more PSs in a linked haplotype for any of haplotypes (1)-(3) in Table 1; or (e) a set of one or more PSs in a substitute haplotype for any of haplotypes (1)-(3) in Table 1. Preferably, the kit comprises a set of one or more oligonucleotides designed for identifying at least one of the alleles at each PS in a set of one or more PSs, wherein the set of one or more PSs is any of (a) PS3; (b) PS2 and PS3; (c) PS1, PS2, and PS3; (d) a set of one or more PSs in a linked haplotype for any of haplotypes (1)-(3) in Table 1; and (e) a set of one or more PSs in a substitute haplotype for any of haplotypes (1)-(3) in Table 1.

In a preferred embodiment of the kit of the invention, the set of one or more oligonucleotides is designed for identifying both alleles at each PS in the set of one or more PSs. In another preferred embodiment, the individual is Caucasian. In another preferred embodiment, the kit further comprises a manual with instructions for (a) performing one or more reactions on a human nucleic acid sample to identify the allele or alleles present in the individual at each PS in the set of one or more PSs, and (b) determining if the individual has a progression marker I or a progression marker II based on the identified allele or alleles. In another preferred embodiment, the linkage disequilibrium between a linked haplotype for any of haplotypes (1)-(3) in Table 1 and any of haplotypes (1)-(3) in Table 1 has a delta squared value selected from the group consisting of at least 0.75, at least 0.80, at least 0.85, at least 0.90, at least 0.95, and 1.0. In yet another preferred embodiment, the linkage disequilibrium between an allele at a substituting PS and an allele at a substituted PS for any of haplotypes (1)-(3) in Table 1 has a delta squared value selected from the group consisting of at least 0.75, at least 0.80, at least 0.85, at least 0.90, at least 0.95, and 1.0.

As used herein, an “oligonucleotide” is a probe or primer capable of hybridizing to a target region that contains, or that is located close to, a PS of interest. Preferably, the oligonucleotide has less than about 100 nucleotides. More preferably, the oligonucleotide is 10 to 35 nucleotides long. Even more preferably, the oligonucleotide is between 15 and 30, and most preferably, between 20 and 25 nucleotides in length. The exact length of the oligonucleotide will depend on the nature of the genomic region containing the PS as well as the genotyping assay to be performed and is readily determined by the skilled artisan.

The oligonucleotides used to practice the invention may be comprised of any phosphorylation state of ribonucleotides, deoxyribonucleotides, and acyclic nucleotide derivatives, and other functionally equivalent derivatives. Alternatively, oligonucleotides may have a phosphate-free backbone, which may be comprised of linkages such as carboxymethyl, acetamidate, carbamate, polyamide (peptide nucleic acid (PNA)) and the like (Varma, in MOLECULAR BIOLOGY AND BIOTECHNOLOGY, A COMPREHENSIVE DESK REFERENCE, Meyers, ed., pp. 617-20, VCH Publishers, Inc., 1995). Oligonucleotides of the invention may be prepared by chemical synthesis using any suitable methodology known in the art, or may be derived from a biological sample, for example, by restriction digestion. The oligonucleotides may be labeled, according to any technique known in the art, including use of radiolabels, fluorescent labels, enzymatic labels, proteins, haptens, antibodies, sequence tags and the like.

Oligonucleotides of the invention must be capable of specifically hybridizing to a target region of a polynucleotide containing a desired locus. As used herein, specific hybridization means the oligonucleotide forms an anti-parallel double-stranded structure with the target region under certain hybridizing conditions, while failing to form such a structure when incubated with another region in the polynucleotide or with a polynucleotide lacking the desired locus under the same hybridizing conditions. Preferably, the oligonucleotide specifically hybridizes to the target region under conventional high stringency conditions.

A nucleic acid molecule such as an oligonucleotide or polynucleotide is said to be a “perfect” or “complete” complement of another nucleic acid molecule if every nucleotide of one of the molecules is complementary to the nucleotide at the corresponding position of the other molecule. A nucleic acid molecule is “substantially complementary” to another molecule if it hybridizes to that molecule with sufficient stability to remain in a duplex form under conventional low-stringency conditions. Conventional hybridization conditions are described, for example, in MOLECULAR CLONING, A LABORATORY MANUAL, 2^{nd} ed., Sambrook et al., Cold Spring Harbor Press, Cold Spring Harbor, N.Y., 1989, and in NUCLEIC ACID HYBRIDIZATION, A PRACTICAL APPROACH, Haymes et al., IRL Press, Washington, D.C., 1985. While perfectly complementary oligonucleotides are preferred for detecting polymorphisms, departures from complete complementarity are contemplated where such departures do not prevent the molecule from specifically hybridizing to the target region. For example, an oligonucleotide primer may have a non-complementary fragment at its 5′ end, with the remainder of the primer being complementary to the target region. Alternatively, non-complementary nucleotides may be interspersed into the probe or primer as long as the resulting probe or primer is still capable of specifically hybridizing to the target region.

Preferred oligonucleotides of the invention, useful in determining if an individual has a progression marker I or progression marker II, are allele-specific oligonucleotides. As used herein, the term allele-specific oligonucleotide (ASO) means an oligonucleotide that is able, under sufficiently stringent conditions, to hybridize specifically to one allele of a gene, or other locus, at a target region containing a PS while not hybridizing to the corresponding region in another allele(s). As understood by the skilled artisan, allele-specificity will depend upon a variety of readily optimized stringency conditions, including salt and formamide concentrations, as well as temperatures for both the hybridization and washing steps. Examples of hybridization and washing conditions typically used for ASO probes are found in Kogan et al., “Genetic Prediction of Hemophilia A” in PCR PROTOCOLS, A GUIDE TO METHODS AND APPLICATIONS, Academic Press, 1990, and Ruaño et al., Proc. Natl. Acad. Sci. USA 87:6296-300 (1990). Typically, an ASO will be perfectly complementary to one allele while containing a single mismatch for another allele.

Allele-specific oligonucleotides of the invention include ASO probes and ASO primers. ASO probes which usually provide good discrimination between different alleles are those in which a central position of the oligonucleotide probe aligns with the polymorphic site in the target region (e.g., approximately the 7^{th} or 8^{th} position in a 15mer, the 8^{th} or 9^{th} position in a 16mer, and the 10^{th} or 11^{th} position in a 20mer). An ASO primer of the invention has a 3′ terminal nucleotide, or preferably a 3′ penultimate nucleotide, that is complementary to only one of the nucleotide alleles of a particular SNP, thereby acting as a primer for polymerase-mediated extension only if that nucleotide allele is present at the PS in the sample being genotyped. ASO probes and primers hybridizing to either the coding or noncoding strand are contemplated by the invention. ASO probes and primers listed below use the appropriate nucleotide symbol (R=G or A, Y=T or C, M=A or C, K=G or T/U, S=G or C, and W=A or T/U; WIPO standard ST.25) at the position of the PS to represent that the ASO contains either of the two alternative allelic variants observed at that PS.

A preferred ASO probe for detecting the alleles at each of PS1, PS2, and PS3 is listed in Table 4. Additionally, detection of the alleles at each of PS1, PS2, and PS3 could be accomplished by utilization of the complement of these ASO probes.

A preferred ASO forward and reverse primer for detecting the alleles at each of PS1, PS2, and PS3 is listed in Table 4.

TABLE 4

Preferred ASOs for Detecting Alleles at PSs in Haplotypes
Comprising Preferred Embodiments of Progression Markers I
and Progression Markers II¹

| | | |
|---|---|---|
| ASO Probe | ASO Forward Primer | ASO Reverse Primer |

| | | | | | | |
|---|---|---|---|---|---|---|
| | | SEQ | | SEQ | | SEQ |
| | Nucleotide | ID | | ID | Nucleotide | ID |
| PS | sequence | NO. | Nucleotide sequence | NO. | sequence | NO. |

| | | | | | | |
|---|---|---|---|---|---|---|
| 1 | GCCTCCCSGC | 2 | GGGAAGGCCTCC | 5 | CCCCCACCCGT | 8 |
| | ACGGG | | CSG | | GCSG | |
| 2 | CTTGCCCYTA | 3 | TTTCTCCTTGCCC | 6 | AGCTCGGGCCC | 9 |
| | GGGCC | | YT | | TARG | |
| 3 | AGACAAGRGA | 4 | AGTCAAAGACAA | 7 | CCTGTCCTGCAT | 10 |
| | TGCAG | | GRG | | CYC | |

¹These ASO probes and primers include the appropriate nucleotide symbol, Y = T or C, R = G or A, M = A or C, K = G or T/U, W = A or T/U, and S = G or C (World Intellectual Property Organization Handbook on Industrial Property Information and Documentation IPO Standard ST.25 (1998), Appendix 2, Table 1), at the position of the PS to represent that the ASO contains one of the two alternative polymorphisms observed at that position.

Other oligonucleotides useful in practicing the invention hybridize to a target region located one to several nucleotides downstream of a PS in a response marker. Such oligonucleotides are useful in polymerase-mediated primer-extension methods for detecting an allele at one of the PSs in the markers described herein and therefore such oligonucleotides are referred to herein as “primer-extension oligonucleotides.” In a preferred embodiment, the 3′-terminus of a primer-extension oligonucleotide is a deoxynucleotide complementary to the nucleotide located immediately adjacent to the PS. A particularly preferred forward and reverse primer-extension oligonucleotide for detecting the alleles at each of PS1, PS2, and PS3 is listed in Table 5. Termination mixes are chosen to terminate extension of the oligonucleotide at the PS of interest, or one base thereafter, depending on the alternative nucleotides present at the PS.

TABLE 5

Preferred Primer Extension Oligos for Detecting
Alleles at PSs in Haplotypes Comprising
Preferred Embodiments of Progression Markers I
and Progression Markers II

| | |
|---|---|
| Foward | Reverse |
| Primer Extension | Primer Extension |

| | | | | |
|---|---|---|---|---|
| PS | Sequence | SEQ ID NO. | Sequence | SEQ ID NO. |

| | | | | |
|---|---|---|---|---|
| 1 | AAGGCCTCCC | 11 | CCACCCGTGC | 14 |
| 2 | CTCCTTGCCC | 12 | TCGGGCCCTA | 15 |
| 3 | CAAAGACAAG | 13 | GTCCTGCATC | 16 |

In some embodiments, the oligonucleotides in a kit of the invention have different labels to allow probing of the identity of nucleotides or nucleotide pairs at two or more PSs simultaneously.

The oligonucleotides in a kit of the invention may also be immobilized on or synthesized on a solid surface such as a microchip, bead, or glass slide (see, e.g., WO 98/20020 and WO 98/20019). Such immobilized oligonucleotides may be used in a variety of polymorphism detection assays, including but not limited to probe hybridization and polymerase extension assays. Immobilized oligonucleotides useful in practicing the invention may comprise an ordered array of oligonucleotides designed to rapidly screen a nucleic acid sample for polymorphisms in multiple genes at the same time.

Kits of the invention may also contain other components such as hybridization buffer (e.g., where the oligonucleotides are to be used as allele-specific probes) or dideoxynucleotide triphosphates (ddNTPs; e.g., where the alleles at the polymorphic sites are to be detected by primer extension). In a preferred embodiment, the set of oligonucleotides consists of primer-extension oligonucleotides. The kit may also contain a polymerase and a reaction buffer optimized for primer-extension mediated by the polymerase. Preferred kits may also include detection reagents, such as biotin- or fluorescent-tagged oligonucleotides or ddNTPs and/or an enzyme-labeled antibody and one or more substrates that generate a detectable signal when acted on by the enzyme. It will be understood by the skilled artisan that the set of oligonucleotides and reagents for performing the genotyping or haplotyping assay will be provided in separate receptacles placed in the container if appropriate to preserve biological or chemical activity and enable proper use in the assay.

In a particularly preferred embodiment, each of the oligonucleotides and all other reagents in the kit have been quality tested for optimal performance in an assay for determining the alleles at a set of PSs comprising a progression marker I or progression marker II.

The methods and kits of the invention are useful for helping physicians make decisions about how to treat an individual. They can be used to predict the progression of AD in an individual having AD, thereby permitting the individual's physician to prescribe an appropriate treatment regimen.

Thus, the invention provides a method for predicting the progression of AD in an individual having AD. The method comprises determining whether the individual has a progression marker I or a progression marker II, and making a prediction based on the results of the determining step. The determination of the progression marker present in an individual can be made using one of the direct or indirect methods described herein. In some preferred embodiments, the determining step comprises identifying for one or both copies of the genomic locus present in the individual the identity of the nucleotide or nucleotide pair at the set of PSs comprising the selected response marker. Alternatively, the determining step may comprise consulting a data repository that states the individual's copy number for the haplotypes comprising one of the progression markers I or progression markers II. The data repository may be the individual's medical records or a medical data card. In preferred embodiments, the individual is Caucasian.

In some embodiments, if the individual is determined to have a progression marker I, then the prediction is that the individual will exhibit a slower progression of AD than an individual not having a progression marker I, and if the individual is determined to have a progression marker II, then the prediction is that the individual will exhibit a faster progression of AD than an individual not having a progression marker II.

Further, in performing any of the methods described herein which require information on the haplotype content of the individual (i.e., the haplotypes and haplotype copy number present in the individual for the polymorphic sites in haplotypes comprising a progression marker I or a progression marker II) or which require knowing if a progression marker I or a progression marker II is present in the individual, the individual's NTRK2 haplotype content or response marker may be determined by consulting a data repository such as the individual's patient records, a medical data card, a file (e.g., a flat ASCII file) accessible by a computer or other electronic or non-electronic media on which information about the individual's NTRK2 haplotype content or response marker can be stored. As used herein, a medical data card is a portable storage device such as a magnetic data card, a smart card, which has an on-board processing unit and which is sold by vendors such as Siemens of Munich Germany, or a flash-memory card. The medical data card may be, but does not have to be, credit-card sized so that it easily fits into pocketbooks, wallets and other such objects carried by the individual. The medical data card may be swiped through a device designed to access information stored on the data card. In an alternative embodiment, portable data storage devices other than data cards can be used. For example, a touch-memory device, such as the “i-button” produced by Dallas Semiconductor of Dallas, Tex. can store information about an individual's NTRK2 haplotype content or response marker, and this device can be incorporated into objects such as jewelry. The data storage device may be implemented so that it can wirelessly communicate with routing/intelligence devices through IEEE 802.11 wireless networking technology or through other methods well known to the skilled artisan. Further, as stated above, information about an individual's haplotype content or response marker can also be stored in a file accessible by a computer; such files may be located on various media, including: a server, a client, a hard disk, a CD, a DVD, a personal digital assistant such as a Palm Pilot, a tape, a zip disk, the computer's internal ROM (read-only-memory) or the internet or worldwide web. Other media for the storage of files accessible by a computer will be obvious to one skilled in the art.

Any or all analytical and mathematical operations involved in practicing the methods of the present invention may be implemented by a computer. For example, the computer may execute a program that assigns NTRK2 haplotype pairs and/or a progression marker I or a progression marker II to individuals based on genotype data inputted by a laboratory technician or treating physician. In addition, the computer may output the predicted progression of AD following input of the individual's NTRK2 haplotype content or progression marker, which was either determined by the computer program or input by the technician or physician. Data on which progression markers were detected in an individual may be stored as part of a relational database (e.g., an instance of an Oracle database or a set of ASCII flat files) containing other clinical and/or haplotype data for the individual. These data may be stored on the computer's hard drive or may, for example, be stored on a CD ROM or on one or more other storage devices accessible by the computer. For example, the data may be stored on one or more databases in communication with the computer via a network.

It is also contemplated that the above described methods and compositions of the invention may be utilized in combination with identifying genotype(s) and/or haplotype(s) for other genomic regions.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims that follow the examples.

### EXAMPLES

The Examples herein are meant to exemplify the various aspects of carrying out the invention and are not intended to limit the scope of the invention in any way. The Examples do not include detailed descriptions for conventional methods employed, such as in the synthesis of oligonucleotides or polymerase chain reaction. Such methods are well known to those skilled in the art and are described in numerous publications, for example, MOLECULAR CLONING: A LABORATORY MANUAL, 2^{nd} ed., supra.

### Example 1

This example illustrates the clinical and biochemical characterization of selected individuals in a cohort of 449 Caucasian patients diagnosed with Alzheimer's Disease.

The patient cohort was selected from patients participating in three clinical trials of galantamine (GAL-INT2, GAL-USA10, and GAL-INT1), and from patients participating in a non-galantamine clinical trial, but with a similar disease population as the galantamine trials (SAB-USA-25) (Rockwood et al., supra; Tariot et al., supra; Wilcock et al., supra). In brief, the trials were carried out by delivering to patients drug or placebo at daily dosages of 8 mg, 16 mg, 24 mg, or 32 mg depending on the trial. Following 3, 5, 6 or 12 months of treatment in the GAL-INT2, GAL-USA10, GAL-INT1 and SAB-USA25 trials, respectively, the severity of symptoms in patients were evaluated using the cognitive subscale of the Alzheimer's Disease Assessment Scale (ADAS-cog) (Rosen et al., supra; Rockwood et al., supra; Tariot et al., supra; Wilcock et al., supra). The ADAS-cog measures cognitive function, including spoken language ability, comprehension of spoken language, recall of test instructions, word-finding difficulty in spontaneous speech, following commands, naming objects and fingers, constructional praxis, ideational praxis, orientation, word-recall task and word-recognition task (Alzheimer's Insights Online, supra).

For the clinical association study described in Example 2 below, 141 placebo patients were selected and used to populate two groups in a tailed sampling strategy, intended to enrich alleles correlating with disease progression in the population. This population consisted of 89 placebo “responders” and 52 placebo “non-responders.” Patients were assigned to responder and non-responder groups based on having a change in ADAS-cog score (ΔADAS-cog) that met a cut-off value that was chosen based on the differences in treatment times in the four clinical trials described above. This can be seen below in Table 6. Table 7 below shows the number of placebo patients from each of the four clinical trials that were placed in each of the clinical association analyses groups.

TABLE 6
ΔADAS-cog Used to Select Patients for Placebo
Responder and Non-Responder Groups

| | Treatment | | |
|---|---|---|---|
| | Time | | |
| Clinical Trial | (months) | Responder | Non-responder |
| GAL-INT2 | 3 | Δ ≦ −2 | Δ ≧ 3 |
| GAL-USA10 | 5 | Δ ≦ −3 | Δ ≧ 5 |
| GAL-INT1 | 6 | Δ ≦ −2 | Δ ≧ 6 |
| SAB-USA25 | 12 | Δ ≦ 1 | Δ ≧ 12 |

TABLE 7
Composition of the Placebo Group

| | Placebo Group | | |
|---|---|---|---|
| | | Non- | |
| Trial Name | Responders | Responders | Total |
| GAL-INT1 | 2 | 0 | 2 |
| GAL-INT2 | 21 | 0 | 21 |
| GAL-USA10 | 39 | 37 | 76 |
| SAB-USA25 | 27 | 15 | 42 |
| TOTAL | 89 | 52 | 141 |

### Example 2

This example illustrates genotyping of the patient cohort for the three NTRK2 polymorphic sites selected by the inventors herein for analysis.

Genomic DNA samples were isolated from blood samples obtained from each member of the cohort and genotyped at each of PS1—PS3 (Table 2) using the MassARRAY technology licensed from Sequenom (San Diego, Calif.). In brief, this genotyping technology involves performing a homogeneous MassEXTEND assay (hME), in which an initial polymerase chain reaction is followed by an allele-specific oligonucleotide extension reaction in the same tube or plate well, and then detecting the extended oligonucleotide by MALDI-TOF mass spectrometry.

For each of the three NTRK2 polymorphic sites of interest, a genomic DNA sample was amplified in a 8.0 μL multiplexed PCR reaction consisting of 2.5 ng genomic DNA (0.3 ng/μL), 0.85 μL 10× reaction buffer, 0.32 units Taq Polymerase, up to five sets of 0.4 pmol each of forward PCR primer (5′ to 3′) and reverse PCR primer (3′ to 5′) and 1.6 mmol each of dATP, dCTP, dGTP and dTTP. A total of two reactions were performed comprising the following polyrnorphic site groups: (1) PS1; and (2) PS2 and PS3. Forward and Reverse PCR primers used for each of the three NTRK2 polymorphic sites consisted of a 10 base universal tag (5′-AGCGGATAAC-3′; SEQ ID NO:17) followed by one of the NTRK2-specific sequences shown in Tables 8A and 8B below:

TABLE 8A

Forward PCR NTRK2-specific Primer
Sequences used in hME Assays

| | | |
|---|---|---|
| PS1 | AGCGGATAACATCCAGGACGACATCCCTAG | (SEQ ID NO:18) |
| PS2 | AGCGGATAACAGTTCCTGGAGCTTTTCCTC | (SEQ ID NO:19) |
| PS3 | AGCGGATAACAACTGACAGATGACCCTGTC | (SEQ ID NO:20) |

TABLE 8B

Reverse PCR NTRK2-specific Primer
Sequences used in hME Assays

| | | |
|---|---|---|
| PS1 | AGCGGATAACAGTTAAGAGAGCCGCAAGCG | (SEQ ID NO:21) |
| PS2 | AGCGGATAACCATTCACTTCCAGGACCTAC | (SEQ ID NO:22) |
| PS3 | AGCGGATAACTAGGTCCTGGAAGTGAATGC | (SEQ ID NO:23) |

PCR thermocycling conditions were: initial denaturation of 95° C. for 15 minutes followed by 45 cycles of 94° C. for 20 seconds, 56° C. for 30 seconds and 72° C. for 1 minute followed by a final extension of 72° C. for 3 minutes. Following the final extension, unincorporated deoxynucleotides were degraded by adding 0.48 units of Shrimp Alkaline Phosphatase (SAP) to the PCR reactions and incubation for 20 minutes at 37° C. followed by 5 minutes at 85° C. to inactivate the SAP.

Template-dependent primer extension reactions were then performed on the multiplexed PCR products by adding a 2.0 μL volume of an hME cocktail consisting of 720 pmol each of three dideoxynucleotides and 720 pmol of one deoxynucleotide, 8.6 pmol of an extension primer, 0.2 μL of 5× Thermosequenase Reaction Buffer, and NanoPure grade water. The thermocycling conditions for the mass extension reaction were: initial denaturation for 2 minutes at 94° C. followed by 40 cycles of 94° C. for 5 seconds, 40° C. for 5 seconds and 72° C. for 5 seconds. Extension primers used to genotype each of the three NTRK2 polymorphic sites are shown in Table 9 below:

TABLE 9

Extension Primers for Genotyping NTRK2
Polymorphic Sites

| | | |
|---|---|---|
| PS1 | CTTTCCCCCACCCGTGC | (SEQ ID NO:24) |
| PS2 | CTCCTTTTTCTCCTTGCCC | (SEQ ID NO:25) |
| PS3 | GATGACCCTGTCCTGCATC | (SEQ ID NO:26) |

The extension products were desalted prior to analysis by mass spectrometry by mixing them with AG50X8 NH₄OAc cation exchange resin. The desalted multiplexed extension products were applied onto a SpectroCHIP™ using the SpectroPOINT™ 24 pin applicator tool as per manufacturer's instructions (Sequenom Industrial Genomics, Inc. San Diego, Calif.). The SpectroChip™ was loaded into a Bruker Biflex III™ linear time-of flight mass spectrometer equipped with a SCOUT 384 ion source and data was acquired using XACQ 4.0, MOCTL 2.1, AutoXecute 4.2 and XMASS/XTOF 5.0.1 software on an Ultra 5™ work station (Sun Microsystems, Palo Alto Calif.). Mass spectrometry data was subsequently analyzed on a PC running Windows NT 4.0 (Microsoft, Seattle Wash.) with SpectroTYPER™ genotype calling software (Sequenom Industrial Genomics, Inc. San Diego, Calif.).

### Example 3

This example illustrates the deduction of haplotypes from the NTRK2 genotyping data generated in Example 2.

Haplotypes were estimated from the unphased genotypes using a computer-implemented algorithm for assigning haplotypes to unrelated individuals in a population sample, essentially as described in WO 01/80156 (Genaissance Pharmaceuticals, Inc., New Haven, Conn.). In this method, haplotypes are assigned directly from individuals who are homozygous at all sites or heterozygous at no more than one of the variable sites. This list of haplotypes is then used to deconvolute the unphased genotypes in the remaining (multiply heterozygous) individuals.

A quality control analysis was performed on the deduced haplotypes, which included analysis of the frequencies of the haplotypes and individual SNPs therein for compliance with principles of Hardy-Weinberg equilibrium.

### Example 4

This example illustrates analysis of the NTRK2 haplotypes in Table 1 for association with individuals' progression of AD.

The statistical analyses compared ΔADAS-cog in patients with two copies vs. one or zero copies, or zero copies vs. one or two copies (within a patient's genome) of a particular allele, using a logistic regression analysis on two-degrees of freedom to associate progression of AD with a particular haplotype. The following covariates were also included: age, gender, history, smoking, ADAS-cog baseline, dose (BID), body mass index, and CYP2D6. The logistic regression included assessment of associations between the haplotypes and the binary outcome of progression of AD.

For the results obtained on the analyses, adjustments were made for multiple comparisons, using a permutation test (MULTIVARIATE PERMUTATION TESTS: WITH APPLICATIONS IN BIOSTATISTICS, Pesarin, John Wiley and Sons, New York, 2001). In this test, a sub-haplotype's data for each observation were kept constant, while all the remaining variables (outcome and covariates) were randomly permuted so that covariates always stayed with the same outcome. The permutation model was fitted for each of the several haplotypes, and the lowest p-value was kept. In total, 1000 permutations were done. Three NTRK2 haplotypes of at least one polymorphism were identified that show a correlation with an individual's progression of AD. These NTRK2 haplotypes are shown above in Table 1, and the unadjusted (“raw”) and adjusted (“perm.”) p-values for these three haplotypes are shown below in Table 10.

TABLE 10
NTRK2 Haplotypes Having Association with Progression of Alzheimer's Disease

| | | | | | Subject Count | | | | |
|---|---|---|---|---|---|---|---|---|---|
| | | | | | for Haplotype | | | Lower | |
| | | | Subject Count | | with Highest | | | Confidence | |
| | | | for Haplotype | | Level Response | | Odds Ratio | Interval (C.I.) | Upper C.I. |
| Haplotype | Perm. p | Raw p | (# of copies) | | (# of copies) | | (O.R.) | of O.R. | of O.R. |
| (1) | 0.023 | 0.007553 | 108 | (2) | 19 | (0 or 1) | 0.243292 | 0.086248 | 0.686289 |
| | | | 33 | (0 or 1) | 33 | (2) | | | |
| (2) | 0.045 | 0.013007 | 11 | (1 or 2) | 51 | (0) | 0.049138 | 0.004558 | 0.529801 |
| | | | 130 | (0) | 1 | (1 or 2) | | | |
| (3) | 0.045 | 0.013007 | 11 | (1 or 2) | 51 | (0) | 0.049138 | 0.004558 | 0.529801 |
| | | | 130 | (0) | 1 | (1 or 2) | | | |

As seen in Table 10, each of the three haplotypes shows a correlation with an individual's progression of AD. When p-values were adjusted for multiple comparisons, haplotype (1) showed the strongest correlation. The odds ratio (O.R.) column indicates the likelihood that (a) an individual with one or two copies of a particular haplotype will exhibit a slower progression of AD as compared to an individual with zero copies of that haplotype (in this “dominant”model, an O.R. greater than 1 indicates that an individual with one or two copies is less likely to exhibit a slower progression of AD than an individual with zero copies, and an O.R. less than 1 indicates that an individual with one or two copies is more likely to exhibit a slower progression of AD than an individual with zero copies), or (b) an individual with two copies of a particular haplotype will exhibit a slower progression of AD as compared to an individual with one or zero copies of that haplotype (in this “recessive”model, an O.R. greater than 1 indicates that an individual with two copies is less likely to exhibit a slower progression of AD than an individual with one or zero copies, and an O.R. less than 1 indicates that an individual with two copies is more likely to exhibit a slower progression of AD than an individual with one or zero copies).

In view of the above, it will be seen that the several advantages of the invention are achieved and other advantageous results attained. As various changes could be made in the above methods and compositions without departing from the scope of the invention, it is intended that all matter contained in the above description and shown in the accompanying drawings shall be interpreted as illustrative and not in a limiting sense.

All references cited in this specification, including patents and patent applications, are hereby incorporated in their entirety by reference. The discussion of references herein is intended merely to summarize the assertions made by their authors and no admission is made that any reference constitutes prior art. Applicants reserve the right to challenge the accuracy and pertinence of the cited references.

1. A method for determining whether an individual has a progression marker I or a progression marker II, the method comprising:
determining whether the individual has (a) two copies, or one or zero copies of any of (i) haplotype (1) in Table 1, (ii) a linked haplotype for haplotype (1) in Table 1, and (iii) a substitute haplotype for haplotype (1) in Table 1, or (b) one or two copies, or zero copies of any of (i) haplotypes (2)-(3) in Table 1, (ii) a linked haplotype for any of haplotypes (2)-(3) in Table 1, and (iii) a substitute haplotype for any of haplotypes (2)-(3) in Table 1, wherein the polymorphic sites (PSs) in haplotypes (1)-(3) in Table 1 correspond to the following nucleotide positions in SEQ ID NO:1: PS1, 2402; PS2, 2722; and PS3, wherein the individual has a progression marker I if the individual has (a) two copies of any of (i) haplotype (1) in Table 1, (ii) a linked haplotype for haplotype (1) in Table 1, and (iii) a substitute haplotype for haplotype (1) in Table 1, or (b) one or two copies of any of (i) haplotypes (2)-(3) in Table 1, (ii) a linked haplotype for any of haplotypes (2)-(3) in Table 1, and (iii) a substitute haplotype for any of haplotypes (2)-(3) in Table 1, and the individual has a progression marker II if the individual has (a) one or zero copies of any of (i) haplotype (1) in Table 1, (ii) a linked haplotype for haplotype (1) in Table 1, and (iii) a substitute haplotype for haplotype (1) in Table 1, or (b) zero copies of any of (i) haplotypes (2)-(3) in Table 1, (ii) a linked haplotype for any of haplotypes (2)-(3) in Table 1, and (iii) a substitute haplotype for any of haplotypes (2)-(3) in Table 1.

2. The method of claim 1, wherein the determining step comprises obtaining the individual's genotype for each PS in the set of PSs comprising any of (a) haplotypes (1)-(3) in Table 1, (b) a linked haplotype for any of of haplotypes (1)-(3) in Table 1, and (c) a substitute haplotype for any of haplotypes (1)-(3) in Table 1, and using the results of the obtaining step to identify the pair of haplotypes for the set of PSs.

3. The method of claim 2, wherein the individual's genotype for the set of PSs is obtained by any of (a) a primer extension assay; (b) an allele-specific PCR assay; (c) a nucleic acid amplification assay; (d) a hybridization assay; (e) a mismatch-detection assay; (f) an enzymatic nucleic acid cleavage assay; and (g) a sequencing assay.

4. The method of claim 1, wherein the determining step comprises consulting a data repository that provides information on the individual's copy number for any of haplotypes (1)-(3) in Table 1, a linked haplotype for any of haplotypes (1)-(3) in Table 1, and a substitute haplotype for any of haplotypes (1)-(3) in Table 1.

5. The method of claim 4, wherein the data repository is the individual's medical records or a medical data card.

6. The method of claim 1, wherein the method comprises determining whether an individual has two copies, or one or zero copies of any of (a) haplotype (1) in Table 1, (b) a linked haplotype for haplotype (1) in Table 1, and (c) a substitute haplotype for haplotype (1) in Table 1.

7. The method of claim 6, wherein the method comprises determining whether an individual has two copies, or one or zero copies of haplotype (1) in Table 1.

8. The method of claim 1, wherein the linkage disequilibrium between the linked haplotype and at least one of haplotypes (1)-(3) in Table 1 has a delta squared value selected from the group consisting of at least 0.75, at least 0.80, at least 0.85, at least 0.90, at least 0.95, and 1.0.

9. The method of claim 8, wherein the linked haplotype is for haplotype (1) in Table 1 and the linkage disequilibrium between the linked haplotype and haplotype (1) in Table 1 has a delta squared value of at least 0.95.

10. The method of claim 1, wherein the linkage disequilibrium between the allele at a substituting PS in the substitute haplotype and the allele at a substituted PS in any of haplotypes (1)-(3) in Table 1 has a delta squared value selected from the group consisting of at least 0.75, least 0.80, at least 0.85, at least 0.90, at least 0.95, and 1.0.

11. The method of claim 10, wherein the linkage disequilibrium between the allele at a substituting PS and the allele at a substituted PS in haplotype (1) in Table 1 has a delta squared value of at least 0.95.

12. The method of claim 1, wherein the individual is Caucasian.

13. The method of claim 1, wherein the individual is diagnosed as having a cognitive disorder.

14. A method for assigning an individual to a first progression marker group or a second progression marker group, the method comprising:
determining whether the individual has (a) two copies, or one or zero copies of any of (i) haplotype (1) in Table 1, (ii) a linked haplotype for haplotype (1) in Table 1, and (iii) a substitute haplotype for haplotype (1) in Table 1, or (b) one or two copies, or zero copies of any of (i) haplotypes (2)-(3) in Table 1, (ii) a linked haplotype for any of haplotypes (2)-(3) in Table 1, and (iii) a substitute haplotype for any of haplotypes (2)-(3) in Table 1, wherein the polymorphic sites (PSs) in haplotypes (1)-(3) in Table 1 correspond to the following nucleotide positions in SEQ ID NO:1: PS1, 2402; PS2, 2722; and PS3, 2799; and
assigning the individual to the first progression marker group if the individual has (a) two copies of any of (i) haplotype (1) in Table 1, (ii) a linked haplotype for haplotype (1) in Table 1, and (iii) a substitute haplotype for haplotype (1) in Table 1, or (b) one or two copies of any of (i) haplotypes (2)-(3) in Table 1, (ii) a linked haplotype for any of haplotypes (2)-(3) in Table 1, and (iii) a substitute haplotype for any of haplotypes (2)-(3) in Table 1, and assigning the individual to the second progression marker group if the individual has (a) one or zero copies of any of (i) haplotype (1) in Table 1, (ii) a linked haplotype for haplotype (1) in Table 1, and (iii) a substitute haplotype for haplotype (1) in Table 1, or (b) zero copies of any of (i) haplotypes (2)-(3) in Table 1, (ii) a linked haplotype for any of haplotypes (2)-(3) in Table 1, and (iii) a substitute haplotype for any of haplotypes (2)-(3) in Table 1.

15. The method of claim 14, wherein the determining step comprises obtaining the individual's genotype for each PS in the set of PSs comprising any of (a) haplotypes (1)-(3) in Table 1, (b) a linked haplotype for any of of haplotypes (1)-(3) in Table 1, and (c) a substitute haplotype for any of haplotypes (1)-(3) in Table 1, and using the results of the obtaining step to identify the pair of haplotypes for the set of PSs.

16. The method of claim 15, wherein the individual's genotype for the set of PSs is obtained by any of (a) a primer extension assay; (b) an allele-specific PCR assay; (c) a nucleic acid amplification assay; (d) a hybridization assay; (e) a mismatch-detection assay; (f) an enzymatic nucleic acid cleavage assay; and (g) a sequencing assay.

17. The method of claim 14, wherein the determining step comprises consulting a data repository that provides information on the individual's copy number for any of (a) haplotypes (1)-(3) in Table 1, (b) a linked haplotype for any of haplotypes (1)-(3) in Table 1, and (c) a substitute haplotype for any of haplotypes (1)-(3) in Table 1.

18. The method of claim 17, wherein the data repository is the individual's medical records or a medical data card.

19. The method of claim 14, wherein the method comprises:
determining whether the individual has two copies, or one or zero copies of any of (a) haplotype (1) in Table 1, (b) a linked haplotype for haplotype (1) in Table 1, and (c) a substitute haplotype for haplotype (1) in Table 1; and
assigning the individual to the first progression marker group if the individual has two copies of any of (a) haplotype (1) in Table 1, (b) a linked haplotype for haplotype (1) in Table 1, and (c) a substitute haplotype for haplotype (1) in Table 1, and assigning the individual to the second progression marker group if the individual has one or zero copies of any of (a) haplotype (1) in Table 1, (b) a linked haplotype for haplotype (1) in Table 1, and (c) a substitute haplotype for haplotype (1) in Table 1.

20. The method of claim 19, wherein the method comprises:
determining whether the individual has two copies, or one or zero copies of haplotype (1) in Table 1; and
assigning the individual to the first progression marker group if the individual has two copies of haplotype (1) in Table 1, and assigning the individual to the second progression marker group if the individual has one or zero copies of haplotype (1) in Table 1.

21. The method of claim 14, wherein the individual is Caucasian.

22. The method of claim 14, wherein the individual is diagnosed as having a cognitive disorder.

23. The method of claim 14, wherein the linkage disequilibrium between the linked haplotype and at least one of haplotypes (1)-(3) in Table 1 has a delta squared value selected from the group consisting of at least 0.75, at least 0.80, at least 0.85, at least 0.90, at least 0.95, and 1.0.

24. The method of claim 23, wherein the linked haplotype is for haplotype (1) in Table 1 and the linkage disequilibrium between the linked haplotype and haplotype (1) in Table 1 has a delta squared value of at least 0.95.

25. The method of claim 14, wherein the linkage disequilibrium between the allele at a substituting PS in the substitute haplotype and the allele at a substituted PS in any of haplotypes (1)-(3) in Table 1 has a delta squared value selected from the group consisting of at least 0.75, least 0.80, at least 0.85, at least 0.90, at least 0.95, and 1.0.

26. The method of claim 25, wherein the linkage disequilibrium between the allele at a substituting PS and the allele at a substituted PS in haplotype (1) in Table 1 has a delta squared value of at least 0.95.

27. A kit for determining whether an individual has a progression marker I or a progression marker II, the kit comprising a set of one or more oligonucleotides designed for identifying at least one of the alleles at each polymorphic site (PS) in a set of one or more PSs, wherein the set of one or more PSs comprises: (a) PS3; (b) PS2 and PS3; (c) PS1, PS2, and PS3; (d) a set of one or more PSs in a linked haplotype for any of haplotypes (1)-(3) in Table 1, or (e) a set of one or more PSs in a substitute haplotype for any of haplotypes (1)-(3) in Table 1, wherein the enumerated PSs in sets (a)-(c) correspond to the following nucleotide positions in SEQ ID NO:1: PS1, 2402; PS2, 2722; and PS3, 2799.

28. The kit of claim 27, wherein the kit comprises a set of one or more oligonucleotides designed for identifying at least one of the alleles at each PS in a set of one or more PSs, wherein the set of one or more PSs is any of: (a) PS3; (b) PS2 and PS3; (c) PS1, PS2, and PS3; (d) a set of one or more PSs in a linked haplotype for any of haplotypes (1)-(3) in Table 1, and (e) a set of one or more PSs in a substitute haplotype for any of haplotypes (1)-(3) in Table 1, wherein the enumerated PSs in sets (a)-(c) correspond to the following nucleotide positions in SEQ ID NO:1: PS1, 2402; PS2, 2722; and PS3, 2799.

29. The kit of claim 27, wherein the set of one or more oligonucleotides is designed for identifying both alleles at each PS in the set of one or more PSs.

30. The kit of claim 27, wherein the set of one or more PSs is (a), (d), or (e), wherein if the set is (d), then the linked haplotype is a linked haplotype for haplotype (1) in Table 1, and wherein if the set is (e), then the substitute haplotype is a substitute haplotype for haplotype (1) in Table 1.

31. The kit of claim 30, wherein the set of one or more PSs is (a).

32. The kit of claim 27, wherein the individual is Caucasian.

33. The kit of claim 27, which further comprises a manual with instructions for (a) performing one or more reactions on a human nucleic acid sample to identify the allele or alleles present in the individual at each PS in the set of one or more PSs, and (b) determining if the individual has a progression marker I or a progression marker II based on the identified allele or alleles.

34. The kit of claim 27, wherein the linkage disequilibrium between the linked haplotype and at least one of haplotypes (1)-(3) in Table 1 has a delta squared value selected from the group consisting of at least 0.75, at least 0.80, at least 0.85, at least 0.90, at least 0.95, and 1.0.

35. The kit of claim 27, wherein the set of one or more PSs is (a) or (d), wherein if the set is (d), then the linked haplotype is a linked haplotype for haplotype (1) in Table 1 and the linkage disequilibrium between the linked haplotype and haplotype (1) in Table 1 has a delta squared value of at least 0.95.

36. The kit of claim 27, wherein the linkage disequilibrium between the allele at a substituting PS in the substitute haplotype and the allele at a substituted PS in any of haplotypes (1)-(3) in Table 1 has a delta squared value selected from the group consisting of at least 0.75, at least 0.80, at least 0.85, at least 0.90, at least 0.95, and 1.0.

37. The kit of claim 27, wherein the set of one or more PSs is (a) or (e), wherein if the set is (e), then the substitute haplotype is a substitute haplotype for haplotype (I) in Table 1 and the linkage disequilibrium between the allele at a substituting PS in the substitute haplotype and the allele at a substituted PS in haplotype (1) in Table 1 has a delta squared value of at least 0.95.

38. The kit of claim 27, wherein at least one oligonucleotide in the set of one or more oligonucleotides is an allele-specific oligonucleotide (ASO) probe comprising a nucleotide sequence, wherein the sequence is any of SEQ ID NOS:2-4 and their complements.

39. The kit of claim 38, wherein the set of one or more PSs is (a) and the at least one oligonucleotide in the set of one or more oligonucleotides is a first ASO probe and a second ASO probe, wherein the first ASO probe comprises a nucleotide sequence, wherein the sequence is SEQ ID NO:2 or its complement, wherein S in SEQ ID NO:2 is G, and wherein the second ASO probe comprises a nucleotide sequence, wherein the sequence is SEQ ID NO:2 or its complement, wherein S in SEQ ID NO:2 is C.

40. The kit of claim 27, wherein at least one oligonucleotide in the set of one or more oligonucleotides is a primer-extension oligonucleotide comprising a nucleotide sequence, wherein the sequence is any of SEQ ID NOS:5-16.

41. The kit of claim 40, wherein the set of one or more PSs is (a) and the at least one oligonucleotide in the set of one or more oligonucleotides is a first primer-extension oligonucleotide, wherein the first primer extension oligonucleotide comprises a nucleotide sequence, wherein the sequence is any of SEQ ID NO:11 and SEQ ID NO:14.

42. A method for predicting an individual's progression of Alzheimer's Disease (AD), the method comprising:
determining whether the individual has a progression marker I or a progression marker II; and
making a prediction based on the results of the determining step.

43. The method of claim 42, wherein if the individual is determined to have a progression marker I, then the prediction is that the individual is more likely to exhibit a slower progression of AD than an individual not having a progression marker I, and wherein if the individual is determined to have a progression marker II, then the prediction is that the individual is less likely to exhibit a slower progression of AD than an individual not having a progression marker II.

44. The method of claim 42, wherein the determining step comprises consulting a data repository that states whether the individual has a progression marker I or a progression marker II.

45. The method of claim 44, wherein the data repository is the individual's medical records or a medical data card.


SEQUENCE LISTING (26 sequences)

SEQ ID NO: 1 - DNA, 40439 bp, Homo sapiens
  misc_feature (2402)..(2402): n is ′c′ or ′g′
cgggcaggagctcggcgaggccaggagagccgcgcaccggccgggcagggacgcgcaggcgtcgggcgccgctggttgcgcatctggcgccagagcgcgcccccagcgctgtgcccgccgcgacccgggccatgggctggtgctccggagcgtgccgggcgctctctccccgcgcgggggcaccagagccctcggaagtgtcaggcactgcggtgtattttccccgttctcttttaaatgactgcggaaaaacagattccgagccgcaaaagggaagacggattctcagacaaggcttgcaaatgccccgcagccatcatttaactgcacccgcagaatagttacggtttgtcacccgaccctcccggatcgcctaatttgtccctagtgagaccccgaggctctgcccgcgcctggcttcttcgtagctggatgcatatcgtgctccgggcagcgcgggcgcagggcacgcgttcgcgcacaccctagcacacatgaacacgcgcaaggtacggcaggggagcaagggcttgggatgccctacgtgggggtgggcatcgcggggagtgaatccacgatctgggaatggctacatcccgtcaggttaaaggatgagagaaaatgacgcggcgccacacatcggacaggccagggtggccaccgagcatttagaatacgcatcaggcgcgctcccttcccctcacatgtctctatttcccgggcgtgtgatggcagaaaacggcaggaaaaagacccacttagagagctagaattgtgtttggagactcccttccctctgcaaaagcaccaacaggaagcccctccaaaaccaccgatccccaaataggtggcccggaatgggggcggtgatccttggttaccaatgcagaacacgttttggtgtttacaacatttatttatttttaggatagcttgccctcccttttaggcagggtgggaaaggagaacgcggggagggagaagggagggaggattcctataataagaatcagcgcatttttcagagctgaaccaagcacggtttccatttcaaaaagggagacagcctctaccgcgattgtagaagagactgtggtgtgaattagggaccgggaggcgtcgaacggaggaacggttcatcttagaggtacctggatgtaaatgcacacacacacacagacacacacacgcacgcgcgtgcatgtctacacggccaggatgtgtgcgtgtgtgcgcgcgtgtgtgaactcccacatgctgctgctgtctgcttctggccagtggcaccgatgcctccctcctccctgctcgcccccagattcccctcccctccctggtgcttttgtctggagggtgttatgggtttgtgtgtgtatgagcgtgtgtgtgtttttggatttcagactaattttctggagtttctgcccctgctctgcgtcagccctcacgtcacttcgccagcagtagcagaggcggcggcggcggctcccggaattgggtgggagcaggagcctcgctggctgcttcgctcgcgctctacgcgctcagtccccggcggtagcaggagcctggacccaggcgccggcggcgggcgtgaggcgccggagcccgggtgagcagcgcagatagtgccctcggtcgcctcggccctcactgtctccccctggggcggcctcggctactccccaggtgggacgtgccgcgccacctgcccgcgccaccggcacccagcggccgtggcggattctgcagcatcattcgggggccccgtcgcggagccaaagccgccggcagtctccgcattcccctttaaagggtccttcgcccggcctgtaccatggaatcctgtcttggggaccctttccctacctcccctcccttggcctcaggctcgaagagagagtgggcacactggtggctccagcggcgtcagtgccatcgcggggcaagttgattcctgggccggagctgggcactcatccatccacagtctccgggctggggtcggggtggggatgacgcgagcagagagggagagtgccccaattagtggtgttgggggtcctacgctcagtcttacgcgtgtctgtttgtcctcagcctcgaggtgcataccggacccccattcgcatctaacaaggaatctgcgccccagagagtcccggacgccgccggtcggtgcccggcgcgccgggccatgcagcgacggccgccgcggagctccgagcagcggtagcgcccccctgtaaagcggttcgctatgccgggaccactgtgaaccctgccgcctgccggaacactcttcgctccggaccagctcagcctctgataagctggactcggcacgcccgcaacaagcaccgaggagttaagagagccgcaagcgcagggaaggcctcccngcacgggtgggggaaagcggccggtgcagcgcggggacaggcactcgggctggcactggctgctagggatgtcgtcctggataaggtggcatggacccgccatggcgcggctctggggcttctgctggctggttgtgggcttctggagggccgctttcgcctgtcccacgtcctgcaaatgcagtgcctctcggatctggtgcagcgacccttctcctggcatcgtggcatttccgagattggagcctaacagtgtagatcctgagaacatcaccgaaatgtgagttcctggagcttttcctcctttttctccttgcccntagggcccgagctggccaggtgggtaggtcctggaagtgaatgctgtcccaagagtggggagaagtcaaagacaagngatgcaggacagggtcatctgtcagttacctgctgtttcactggcttgggactgctagtgcagggagaagtgtgacactttagaataagtttcaaatatagatatacatatgccgattatatatttattcctcgtgggaaggaactcactatgctttttatatttcaaagctctgctgggtgtgctttaggctttcagtatgcactaacttagttttgggcatttttgtttcataaagtgctgtgtgtatgttgtgttagaagttccattaatattcactagaaaagaggttaaaataattaactttcccaaatctgttgagggaagtgacagtctctcccttaacatcttttttgagatttaagttaaccaatttcccttttcattttatatatcggattttgcttaggagatggcatccagagatggccttgaaaatgatctgtaagaggagaagttctgagtgttgaaatgcactcgctcaaaagaaaaaaatatgatagttggaagcctaaaagcctggtctaatttaatttggacttgacttcccagagatatgattttgagaccacttggattataactcttaattgcccattgaataatttcccaggactaggttggatgataaagcactgggtagaactctgatgaaggctggctttagcattggacctctgtgtccctggaaatgtttgagcttgttgtggcttgaagaaacagaactgtgtatttctattctccttttctgatgctttgtgacaaatgcatcaccaagcaagaacttgccagtcctttctctagcattcctgtgtcacctctactggactgttattgttatcatttttttcctgaaggatgctgtcattccatacatctcattaacatgagattttattcgtatacgtggaagattgtaaggcaaagtttctcccagttttccttattgtgtttgatactgagataggcagccctaaagtggggcttgggctgtggcctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctgtgatgcatattaagaattataaaacagatctagctatctcaaagacattgtgatctaagaaaactgaatagacaggtacttaaataactgtagtacagggtagagtataggaaattccatgatgatggttttaatagttatttgtttggatcactagggactttactgattgcaaatgatagaacttaagattaacttaggttagtgacatctagcctgagagacagttgtatctaaaggctcaagtggtatttttagagttctgtctctttagctctgcttctttctgtggactgacttcattcttggacagtctatttccttgtagcagaaaatttccccatggaagccccaggcctgtgccatccttcagctatgatcccagagtaagacagagactctctcttccccagcattcatttatttaatctcggggaagactcctcttagtgtaattgagatcatgtgcacatctctaagcccaccaatgtgggcaaagggatggagttttctctctattcacccctatattggagggcttagggggtgggggagggaggaggttcccacggagaatgcgctaaggccatatggggtttgtgaaatgtggttcatcaaaggaaagattgatactgttactgacaaaggcagaaggaggagatgctgggccaggaaaatagagagaaaagaggaaggggtagggaaaacgcaccttagtattttaggaggtaaaggagggagtggctgtctctcttgaaatgtggggaaatatgtgacttttcttgtgcagtgatgacagcagcatctctgtggctctcaccgtaagagtggaaacacctttgagaggagtagatgcatttccacggtgttttcacatatgtacactggtgatactccaaaagggaaagaaaagtgaaattgttgggtgaaagagtgatgaaacacgaggagaaatgaaacccagtgcaagggttattgaccatgaaacctggtcagtcatggtttctgctcagtcatgctcagttttgctctactgctgtatttaaaaaaatggcaaggctcagagtggtgtggagggagcaccttggacacctgggtgaggtggatgggagttcttcatggtgctgctgcctacattctgagtcactgcgattcactctctgctttgttacagtttcatcgcaaaccagaaaaggttagaaatcatcaacgaagatgatgttgaagcttatgtgggactgagaaatctgtgagtactcaggaccagggcacattatctcagagaattttcctgttgtctgctctggtcaggcaggcattcactggttcgttctaatgtgcatgaaattatgtgttttcacaggacaattgtggattctggattaaaatttgtggctcataaagcatttctgaaaaacagcaacctgcagcacatgtaagtagagattgattcttttgcttcccaggacccattttattcaatatttcccccctctgtttattttccttcttttccaacttgaaacctccctttttaaagttagtatagctgtgataatagcttagaaacattagctctgattttgtttgattttttgaatagcttcagaggggtacttagaacaggctgtttattccctttcatgaatttctgggccagttccatcttggacagagcaataggctagcagcaaggagtctgatgtgaaaactgtgtccttgcacagagttcttgattttcttccttggaataatgacatactccctggaagacatcaaactagtggaatcgaaagaagctgacttggccttggcctttatagggaaataaaaattactggtgcttttcccctcacaataggattctcttttgaaccttcattttcccaaagccgtctgtcatggtttgataaaggtagagaaaaatcaatcatgggacatgccaaaggtacagaaaattattaacaatgggaaatatttccccaaagtcttgttgatgctttgtccactgaagtcaaacggaaatatggaaaatgggaactgttagcttgtttgttgctaaatcctctagatgatggcatttaagtccataaggtgtggctttcctgagagttgtcttcattaaactaaagatcacctaagtgcagaatatttatgcatcactgagctcagacttgtactcccatcccctgagtttttagtggaacttctgccagaaggttgttgcctgcatttattgattctgttttgctgtaccaaattctctttgcagtgataccttacatgtcatagtctatctgtgggaccctcacctggattatttcacatgatttttttctgacaattggtaggacagatcttttttatgcacatttcatagataaaataagagtgagagagttctaggacttgctcagattcatagatcgtaagtannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtgcagtggcacaatctctgctcactgcaagctctgcctcccgggttcatgccattctcctgcctcagcctccccagtagttgggactacaggtgcccgccaccgcctggctaatttttagtattttttttagtggagacggggtttcactgtgttagccaggatggtctcgatctcctgacctcatgatccacccacctcggcctcccaaagtgctgggattacaggcgtgagccaccgcacctggccagatctcatgttttatccaactctcaacccaacacttgccttcctcggcaggaaacacacactgaaagtctcattgacttcctccggtttgtagaggtcttggctgtttttatgaaagttgatgtttattttacattcattttatttattaaacacatcttgaggcccaagcatcggatactctttaaataaatttaaacagtaaacaaattcgcagagtgagtcggctgccagggttatggctctgagtttcctaccccaaaatctcatgacactgatcattggaaactcttttatagaatcaaattacatgtttggaacatgtcagtaaattattattttttttagtcacaaatatatctttttattgtctggttggtctaaatctgaagatggtatgtcactagaatggttcaagatgaaatctgacatattccttgttacctaatttttgtttgttctcatcgtggaggtatgcggttatttagatcatatgtaaacaatctcagtagagttttttttatcaataaaatgtctttcctataaagacgtattgaattcagttaatattcagagttttgtatgccacataatttcaaaatatccatgatgctctttagttttaaggcatattttttccctactgggtttatagttttaaattttatttaatcttacatatcctcctattaattgttcaaaagatcagggtttctataagatatataacttggctccaaatcaacatatgaaaagtaagcttttgggtgaaaaagacattaaaaatacacaatatatttatgctacccctcccagcaaagaaaaaattatagacaaggttagaggaaaaactcagctatcactctaagtgaaagagagagagatctggatgtaagtaaagcttatataatgatcaaataaaattatattttgaaaaacaaatctctatttgaatactgtgttcctaaaatgtaacattttaaattcatgtttaatgtttttgattcctttcagcaattttacccgaaacaaactgacgagtttgtctaggaaacatttccgtcaccttgacttgtctgaactgtaagtaatgattttgtgtggcatttggggaaatgttttcaaaggaaggggtaattaagtatttttcttttaatgagtgatgttgcagatctatttttataccaaattctcaaaaaatgatttctcaagtttcagtctctcagtcttttattccttcaaggtttattatctctcatttttaaaattcagtaattaaaaaaaatagaaatctcagtagtgacatatcatttctgattcatttaaatgttgattttacataaataatttctggtacagggaaaaaagagaaaaagaagggggctttgtgtgaaaacggctgtatgctttatatctcacagctctggtttggttcctgttggatccaggccctaaagtcaagcaaacccattcaaggaggaaataaaaatgggcaaaactctgctcacttttgtccatcttaaggataggggacattggaggagaggtagttgttctctctccccacccatctttgtgagattttcttccagatctgttattgcattcaatatttatttttaagtcccattttcttctgttgtaaaatgatagaaaacgtattgaaaggtgttgatgactaacagccaggatttccaagtgagctccattccttgcattgtccccattaaacaaatatgaactgttgctattcttatggtaaggcacctcaaagcatcctaatgcagaggctttgtggggcagatgtgggcccaggaagaggcctttgaagattaggcaatggcatttgcttaaactctatcttgcctgaattgtggagtctttttatgaagatccatccttattcacaaggaaattgtgcagattggaagatgtcactgctggagagacaacaagttaataggtgtaatgggtgttatatggtgaaagtcttggcaattgagaattatttggaataaagtgaattcaggacgttgaagcactaatgcttctaacacaggaataggcatgacaaactaaaggaagatacatgagatagggtaaagtacttggtgtaaaaattttgggcaacattgtgtaaaattttcttgctggattaaaaattagagtttcaatttcaaatttatgtctcaatattcttcttgaagcatatttaatcaattaattcaacaaatgttaattgtgtacctattacaaatcaaaatgatatggcacagggccatggggataaagggagaaaagtttctatgttcctggattctgttatccagtgggagaagtagaaacctaaagataaaagtgtaatgtgctgtcataatcatcgagcttgcatagtaagctggagaaagaagccagctgtgcaccagtgttgtgttgtcatatgaacattgctgtattcataattgaagcattcctttccttctttatcccttctgctgagttaatgatatcttcttttagaaagactctttagttttgcatgtactcagagctccaggaagcattttggattagtggggtttaaactggtggtgggtcttttctgcattctattccatatctcataacatcaaatcccattttggtgttggagggatacttggaggcgatcaagcagacacaaattaaatgagtctaccatgaccacattttcccctaagaaactcctggaaaagtctccagggatccctgagattcttagaacccaaccagaaaaccacaggagtaaatgatgttgcaaagcccaggtcctgtcttcacggaaatcttaagtattgtggttaatgccaggcagagtctatgtaacgattttaaatggcctcatttaaaggctcattaccataaatggaattcatcttaaataggccactgagtattacaaataagcatttaaagatttttttttccttgtggtttttattttcctcttgactacttcctcttttacccattgcaggccacaggagtccaggcagcatggctgaggtctgaatggcggtggctttccacccacagtggtctaacgcagctcaatctgcagctcagttgccttgccctcagaatagcttgctctgtgtgtgtgtgtgtgtgtgtgtgtgtgtgtgtgtgtgtgtgtgaaggaggtctacagcttcctttcaggttctctctctcacctttactccagctatcttgaatcaatgaaactgactgtgattgtataatgcagaaagctaggggtgccagccttctggcattcacaatttagtcttagctttgtacttacagtctcattttaaattgagatgaaattgttctgccccctgctgaagtttgagcttagaaagtggtaacacttgagcctttagcctggtcagaaataagtatgttagcattcctgaaaacttcttgttggcatccttttaataactctataataaccatgataaatttgtcatggcaccttattttccccatgttatgagatgttcttttctatcctatattttggctatttctgtctctatcttgtttcccctgttattctgtaaggtcttctatcattctccagtgctgtctgcatataggagatgctccgtaagtatcagataaatgaaggaataataagtactgtcatgctatgaaagtggtatgacttccaagcattgctggctagaaagaattcataatgttgtagtattttacaagcactaatgtagctaaaatggaaaaaggaacttgatctgttgtcatttttgttccctgtaggatcctggtgggcaatccatttacatgctcctgtgacattatgtggatcaagactctccaagaggctaaatccagtccagacactcaggatttgtactgcctgaatgaaagcagcaagaatattcccctggcaaacctgcagatacccaattgtggtaatttatttttaaatcaatgtgttctagttgctattaattattctcattgccttggtgcggtagtctgggaaaattaccactacctggattgtatatagtattctgttgatgtctccagtgactcagacaacatttatgtgcagtgtggagtaatctttgcaagtagaatattaattcagagtggatggctgtaaagtgcatagagaaaaagtacactgcttattctttttgtttgtttgttcatttgcttatttgttcatatttgtttttccatctatccatccatccatttatccacccatccatctgtgcaaaaactatctgctaaaagtgtggcacttctctggccacatatacccttggatggcacccaggccaaggatctcagggtgatgaaagtgtcctggcagcacaagcaaatccaattcaatccagcacattcccttaaacttgtcttactgccagaggtctgtgtctcttgatggggtgccccaagtctgccatgccacgtgcttctccccatgtctggatttaagatctcctactttaccgtcatcccctattcctcttcccagcgttttcatctccaacatttaaaagggcaccgaatggcaaaatcagagctcatgtgagtgctagtgagttataaggaaggaatagcaaagtgttctgcttaggattttactagtcttcagtgatgtacctctacaatacccacacatgcatgttactaccccattgacatttgaatcggttagtcaaccagttaacaagttcacaaacaattatgtttgatcttctggcctagtggctggtgacacagcagaaagccatgtagcacctggttcttgtgcacaaggaatctgtgtattaatcacaaacaaccgattctttgttttcggaagacgctattatttgaggtctattttttatttaagaaaaatggagctgctgcacacgctttgtaggaatgcgataaaaataaagatatgagtctgtttcttaccctgaatgaccttagaagacggccctgacccttcccacagatttttctgtcacaaatagagtcctttggtttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatagactgtacaaatcttattccagtctgatttcactaataaagaaagatggctttgccggtaagaatacatgataagagaaaggtttatccataataatgtgaagacgaagggtcagatattgccaggtgggtgggttaattgcaatttaacggctgttgaaaatgaatcgccttctttgacctccaggttctgatctgagttaatgatgtatcatagttcttcaagacatagctgtcgggtgactatagtcccaagcagagttcctgacaccaagagagggacaggctccactagcaggtcctgacatattgttataccctctaagacatgcctacgtgtaaaaattaccatgattcttatgcagtttaaagcagtgtccacactcagttggtgtgataatttcatgaagtgatagcagcatccctaaaattacacatagcttttgaaatataaaagtataaaaatgatgctttgctggactattaaaaatgtttatgaggttcaaagtttcatctgataaatacattctcagagtctttccaataaaaagaacttttcactgaaatgagtccaacccattttcattatattccaaacaagatagcaattttacctataatatttaactatgatttatataactttgaaactttctaacactgcaatagttcaaactgcttgtctttgaaaaatgctttgaaactacattttcttcaatagaatgagtctttcaaaatctgtgtctggtagagggttagatttgtgagagttacttaaagcaaaaaacagatttttgcaaaaacaaaataggtgcatctcacccttttgtgttcttgagcattagtaaaaaatggggggtaggagacaggaggaaataaaagacatgatgtcagtccttatgtacaatctctgtttatgtgcaaaaactggattagtaactgtagaacaggaaaagaggtggaagacagttctttatactagtgttcttgaaataaaaggttattcggcaataatgctttaagtaatgtgcataatttattttgaaatctaattatagctttttttggtttatgttagatctatgaagctgatcaaagtagcactttttcagatcaacagaaatcagtctcaaaaagcaattaaagattgatttttaaagcataaacagttttcaatttattgcttttattttgatactgcatttaactatttgcatatgcctctgtttacttttcttgttccataggtttgccatctgcaaatctggccgcacctaacctcactgtggaggaaggaaagtctatcacattatcctgtagtgtggcaggtgatccggttcctaatatgtattgggatgttggtaacctggtttccaaacatatggtaaggcttgtgtttggctgtgtcttaatagagagacaagagtgtttcagaatgcgagaatgtattaaacctagtgatgatcatttgatattttataaggctacatatagaaatttacaaagagtttttgatgtacattcactttctacttattcttaattaataaagactaatgcaggcttgcattttagttatgcatacctaagagagcgaggagtctctatatccctagtgagctctttgtatcatcctgaacaaagcgagtgactttccctgtggtcttctcccttggagggaattcagggatcaagtcaacctcatgcacttagcaattgaaacctagagaaatagaataacttctgatttacttcttcgcctgagcccagttcaggcagagaagcttttctaatgctatatatacatattttctctagttaggggaagaaaccccaaatcttgtcactttgggatcaatcctaatcaaggttatttttgtctgttaattcatttgtagaatgaaacaagccacacacagggctccttaaggataactaacatttcatccgatgacagtgggaagcagatctcttgtgtggcggaaaatcttgtaggagaagatcaagattctgtcaacctcactgtgcattgtacgtaatcagactggcatgtgtttttaatagcaaatgatcatggacgtacctacgtttgttgctggggcactctgggtgctgcttaaatttgttaaaaaaagtatatgcagtgttttgtgcatactctattaagaaacattatcagcctcttttactgttcaatttctgcttaataactcttgctcatgcatgttttttcttttaagcttctttttcgattttgagcttgtcattttgtgccttcctctgtcttttttctgatagaatatactttgggctggttgctccctctggttaatgttttagcttaaattttcgtaaatcagtttctaaaactttcttcctctataaaagcaatatgatgctttaaagttatgatacctaaagtatttactagctgttcacaaatgcctaattgtactgttgataaaataaaacctcattcttttgttatcaagtttctgtgagaaggtaggattagtcctacttaaattactgaaaattattatcaccggtagaaaacttttattattattgttttaaatttctgtatacattgaatctaagttcaaagatatccctctttttaattaaacataagtacattgtttttcagtattgtaactaatacaatatacattttgaaattccaaattatggcttttagcatgaaggtgtatattcagtaaaaactatatagacacttcacgtcattgtaaattctgagctggcacggtataaattaatgaggttttactctatttggattagaatttggatgagaaaaacatgaacatgcatgtgtgtaagtgtatgtctgacttacacacgtgtagacccatggccatagctggtatcaatatgctggttaacactcactcatagcacacattcagtgtgtgattgctggagtgaatgattgaaatcatcagctgtaatcctttatcattgttagtacaatcactatgcccagtgggtactaaactcataatttctcatcattttgacccactcagaagcctgaagtcaatgttatctccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtgccactgcactccagcctgggtggcagagtgagaatctgtctaaaaaaacaaaaaacaaactaaaaatattaacaaccaagataaccagtgcagagaccccactcagagggtttgtctgttccctggctacaataccttagatttttttctcctcttgtttgatgaacacatacagtcaagaagcagtgcctgagagggaagggccactgtattaaacatcaggattcaagattcttgttccagatctgccttgctcagtaaccttgatgagctcacttaacctcattgcactttggtttcttcatctgaaaaaaatgatgctattagatctcaaaggtcttttccagtccttgattttgtaattaccaaagccagactacattaaaatgaacagatgaagcattttaaataccctcacttaggataagtactctgtttaaaaattctacttgataatgtttgtttccccaaagcataaagtgctttatatctaaagttgaaataatccctactctgtccaggcttagattggaccccaaacatttaaaagatctaaacattggcatctaggatcctagcaaaaatggacaacactatcatgaaatgtgatactttcaaagagtatttactattgacaatcagtaataaatgttgatttgttatatttataataattcatcatcagcagcaacatttattgcattgatcagtttgatctgttttcgaagttatgtgagatcagggcttcaggcaaaattttaaaagtcatttttgttatttaggaaagaagtgctatcaaaaagtattcattattttattattttgagtttttttggtgatgaatctgtataatatcaagatacagatattagaaaccaaagtcaacttcaaatgaactctttccgctgtcagaaatctcttggatctgtttggagctggttttcacttatactgaatagatttgagaaatgactgtaatgcaggggtcttaagtagaacagagaaaggagtctctctgaagctctctttggggagttacatccaccctgatgatgatacaaaatactgatggattccagagtttctgatgatgtgtttttctaagccaaacaatggttgagtaaaattccctatcaatgacacagacatctcgagatggggagaattctgagctttctgatgctattaactctctctttttcaatttagttgcaccaactatcacatttctcgaatctccaacctcagaccaccactggtgcattccattcactgtgaaaggcaaccccaaaccagcgcttcagtggttctataacggggcaatattgaatgagtccaaatacatctgtactaaaatacatgttaccaatcacacggagtaccacggctgcctccagctggataatcccactcacatgaacaatggggactacactctaatagccaagaatgagtatgggaaggatgagaaacagatttctgctcacttcatgggctggcctggaattgacgatggtgagtaactgacacttttgtatgtggggagaagataaagtctatcattcacctgttgacaaaatcatgtatacaataagccatcccccaacctagtggcttaaaactccattatttagtgttgctcatggatccataggtcagcggagtggttttattttgcatgtatttattgtggaattcagggtcatgaagcagcagctacataggagatgtgcctgttatgataagtgtggagtgaaagaggacaactgcaacgatgtgaggcttcctaaggcctagattcagggctggtgcactggactgtctcttcagcttatctgccattggccaaagcaagtcacagggccaaagccaagttaaggggcagggagttgtactcctcccaagaaggtgatgaggaaattaataattttgaacagttatctgatacacactggattaataactactccaggatgcttgggtatcttatgagaaaagggttattattattatttccatcactgttgcaagtatttaatgagattttgtacccacaatggcaaaacccatgcaaaaccgcaagcgcaggaactgtgtatggttggctcgtgatgcatttaagatttattttctagcctcttacctaattcaaaaaggagaattctggtttctcatccatggatgagaaattagtgtcttggactgttttgcttacccgggctgactgagctgagtgattcttttattggctccggagctgttttcctaggagtggtaatagtgcttctctggccaaaggctaggactgcggaaaggatggctagcatcaggggtctactctgggcacagcaggagcgtgtcagtgtgagcttcaggaacagtgggacaagtgtcatgcatttacctgcttcagatgctgtccttcagtggagtgttcttacctactcagtctcattgcccaaatgcttgctaccaatgtggtggccaaggcctgtgtttggatgttgggagctgtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntacacagccattcatttcattttttttccctgctaaaatagatatttttctagttgaaatgcgttacaattctaatttgaacactggggccacttttatttggttcttcatttagactaaccttgattattcactggggaatgcatgaacagaaaattggctcatttcgctttactttaatggcattttcctgctaagggttcagaagtgaaatgaaaactgagtaagctttatggtttacaagttttactgtttcacatggcaagtgaggactaaaagcataggaaatagctttggaaattcattttgcaatcacttaaatgttaaaacaaaatttaatttcatttccaagtagactttcatgtagagacccagttagctctgattggagaataagtagcaaatgtttttgttaccagttcttgttgatgctgagttaaaaaaatgtttttggcaagtctcataggaataccttaaagcagaatattttgatcatcttggtgactgcagagccatcctccaaaattaaattgataacttttacatttctctctgacataccattttttaataacttcttacacatcaatgatttcaatagaggaagtacattttatttttctttgttgtgttagggcttaaaaaaggtttcttcagccctagaaactcccctgaagataaggtggagaataaaattgtgctttagatagcattatgcagggtggttaataacaagctcctgaatgcaaatggccctgctacacagttaacagtaaccctcaaaccatcagagaataacaaaagtggtacgaaaattatttgtctctggaataaaaataccgaaactgcaatgaaagtagccaattgtataagagaaacagtaccaaaagaagaaaaatggtaataattcattgattgctgaagttttttcaaagcaatttgtgatctttttttgtgatttgttttcttttttggagggatgaaagtgggagaaccggggaggggaacggagttaatgtggaacaactcaagaacttaaagttttgtttacctgtcctgtgaaagcagtctattaaattgcaaaacttcagtgtatgtctagcttattttagttgacataggttagtaattttttgactccaaaatgcatacttacaaatctttgctctgttttgccttttaggtgcaaacccaaattatcctgatgtaatttatgaaggtagctatcgtgttttctactttgtatttctttttcaaaatgtttgtgtatcatgaagtaaatcaactgaaagaagactcttctaagctcagttaaatttacactttaagcattacataggccaaaatagtatttcaaaaaatcattcaatattgatggccgttttcccttgacatttacagtgcctgtaaatagcactttctctctcataccctgttcttggaagtagtgaatccattccgctggctgtaacagacatcgtatttccttaattgggaactaattacctgtttaaaagccatggctaccaagaaaagggaggtatatcagtttctggctatgacagtaaagccaattcaaaatgatgtttcttgtgtaatgaaggtgtttggctcacagatgggaacctcaaaggtagggcttgatttagggttggttaaagcagcagctccactatgtcatctgggacccaagttcccttcaccctactctctttttcccaccacgccagcttcactgaaggctggtccccttgtggttacaagagaatgtggcgggggagctgctgccattcacaaggatgggagaaagggaagcgctcctgaaggcgtcctcaaggaaagagaacttcccaagagacaagagccaagccctctttgtatccaagtcaagacccaattgggtcatagtccattcttgcccttattactgctaagagggatggattatcccttcagtaactaaattcacccccagactttaagtcagcttccccgaaggtggataggcttaaccccttgaaaaaaaaaatgggattctctcaggaacgtggaaagaaagaaaccaaagtccattatattagtttttttttttttttttttttttttttccgagacggagtctcactttgttgcccaggctggagtccagtggcgcgatctatgctcactgcaacctccgcctcccgagttcaagcgattctcctgcctcagcctcctgannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntatccattagtgtttaaaattaataagttttaagatattcaaaaggaaaatgatgagaatgtggttttggcattaagaatgaaacagcaaacacatagaaaatagatttttttctgttattttttattcacaaaggaacacattgctaataaaaatacaatttaaattggttgccctgaggccagcatgatgattctacctggagaagtttagctggggctgcattaacaaggtagcctcttcaggatggaaattgaaggcagtgtacctatttagaattaaaatacaaacattataaacctcattgtttgcaataagtttaccaagagttttctgagaccttttatatgcaatattgctaataaaaactttgacaacccctgaaaaaaaatcagtcattatttctatcctgacccatgtagactaaaattgatccagaaggcagcaactgcatttgcgacttggtgaaatgagaacagagcgttaacaattctaagtattgtgtggatctaccttttaatcattctgaaactgaaatattggcaattcagaactcttcaaaaaagtttacgtgtggacccaagtttcatactgatgcttgttccacatatcctcttacgtctctgccctttgtgcaaataagttgcacctgtcctggaaatcagtgttgctcatttcaaatctggcgtcacgtgtgagcgctaccatcttcttgccttcacctggtcatgtaactttcctccactgatattcttcagctgtaaaactggtagttatgctctgtctctgaagacctttccgggtctataaatctatgacacgtagatgatcgattccctgtcgtacttgttttaaatatgccatccataaaggggtatatgcttatgtgcaaagaaaaatccctgaaccaggtttggggctgaccaacaagacaatgatgtaaaggtctgggttgaataaaatgtgggcgttgtgcagtctcagcgctgcagtgcattgaactcagcagcctggtcacgtccctccacagccagactgtcgggggtttggaatctttgtgatttgtcttgcttcattctggctcttactgtggccctgtgtttgttggcagcttaatgacaacttcatgttcttcctcattccccctttgcccacttaagattatggaactgcagcgaatgacatcggggacaccacgaacagaagtaatgaaatcccttccacagacgtcactgataaaaccggtcgggaacatctctcggtgagtggaataaataggtgtctgaattggttctgagcattttggatgcctccatgttagaggaatgtagctgcttcaataagacacttttattgttcagatataaatagggtgttaacacctaggttggaagaattaataaaaggcttaacaaaagtgataaaggtgttatatggcctttgacttatttgctgcttttgtgcaccacttttggttgttagtggttcctatttcaattatctgtgctttagaagggtatcaccagaagagcaagaaaccagctttatcattcagggactacaccttcatacctgtctccttaaatttttaatataattttgactgctcgctagatgtagggaatccagtagcttctgaaagggactggcggtggaggggatataatcatatgaacaaaattaagattagttgtttagaattcatcagagaccctggaggtgaatattagcacctcaaatacaaatagaagcagagcaaggcagtacaagagatagaagtggaggagcagatgttttgttccttgtagctgggacactctgaggagcctttgtggggcaagtgactttttgcatggcctgtgcagaagggttgggattttgacatacaggaatggaagagaagggcatttggggcatgtgaacaggagcttgtaggtggagaaacatcgtgaaggtgcttgattggagggtaaggcatgtgcaggacagcagggttggagataaagattgtggccaggttgtaggtcttgacacagagtctcaggtgtgtagatttatggcacagtgaagagccattgaagagggaagtcacatgatcccaggggtctatggtcagttgctctggtccgaacccaacaacttcctccagttgtttcttttcctcactgtcatatgcacacagagcttcctgtgagctctgcctccttataaattccaaatctgtccatatctccttacctccactgccactgtcacaggctaacccaggggttggcaaactatgacccatgggccaagtccagctcaccacctgtttttgtatggttttcaggctaagaatggtttctaccttctaagtggtttaaaacaacaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtgccgctgacaactttgctctaaaatgttaagatgcacttagaggagcatcatgccttggtcagcagcaatggtagcagagtgatcgcatttctatgtgtacctctgtcatctctacctctgggatgggagaggattcacatatatctacatctaccctcttgacttgtgtaatccacacctggcaattacatttttaaacttacttaaacatttttaggcttatatcatatatatgggaaaatgcacatatcataaatttacaacccaatgggctttgacaagtggaactcaccccagaagtgccctcctgctcccttccagtgccactcactgttcccagggtaccctgacttctaagagcaacaatagctaaccttaaaaatagccttttattatggacaattttcaaacatgcacttcagtagagagaatagtaaaatgaaccccattgtattcttcaccagcttcaagaattatcgggctggatgcggtggctcatgcctatattcccagaactttgggaggtgtaggtggaagaattccttgagcccgggatttcaacaccagcctgggcaatatggcaaaaccctgtctctgcaaaaaaacttaaaaatgagccaggtatagtggtgtttacctgtagtcctagctactcaggaggctgaggtgggaagataccttgagcccaggaggtcgaagctgcagtgagccatgattgtgctattgcactttagcctgagcaacaaagtgagactccgtcacacacacacacacacacagaaaaatcaccagtattttgctgttcatatttcatattctctctctccctttctctgcccacctctctttctctctctcctccctctttagatggatggagagagagctgtaatatttttaaaatgccaattggatagccatttttataagctttaacttatgttttctcatttgtaaagtaggcataataatagatcttacatacttcactatagggttgtcgcagattaagtgagttggtgtacataaggctgttataagaacaagtggtaagcattcaataaatatgaactgcctgtatcattatcatcgtcatgatcatcatcaccatatgattatagggaactaattagcaaggttataaccaccctcccttcctttctctcggtctatgctgtggtggtgattgcgtctgtggtgggattttgccttttggtaatgctgtttctgcttaagttggcaagacactccaagtttggcatgaaaggtaagaagggttgtgtttatttagcttcttatgtggatcatttttggcttatgactaatgctaattaccactaaagaaggaagtggctagatttatgatgattaggttttaaggctaaaaaaatagcaacaaggctttgatactacaaatcaagacggagcaaaataatgttgattactcagcactacccacttttccatgagaacagttgtcccgggagacagaactgggttcttgagtatcaagggactaataaagatatgacaaagaaatagtacttgatgtttcttttgctccagaaaggcaggtgagacacagtggggataggataacctggacacatgtattttgtgcagaaaggtctatgctgctgtacatttgattcccaaatcttcagtggtatgcttttggtgttgttcctgcttttccagatcctcattctttgaaatttaaacaacagtgttatgaagcaaaataagaacagctatcagcagtatatagaagcttcgtgaatacatagctttttttttatttaagtggaaaaattagcagggctagattaaaaattaggcactaattgacccaaaatagataacttgtctttttttagaggatggaaaattaattttacagggaaaacttgtataaatttcttgttacataaaaaatgccttggagcagtgatttaatgatttaaaaccatttgctctgaagtttaaacttatgtattggataatatgccagtgttgtattttgtgtggtctcctatagatagacactatttaaaagtgtctaaatgggaatgctgtgaagaaggtttagtggtgtcttagttgtgctggagagtaggtcaggtggattttctttatctgtatggtctgttagaattatgggtaatgttgctaatattgctatttacttgaaagggaatgtttcagtagcaataagttgcccagcatacactgacatctgggatagagcagatctatttctgatgtttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctgtgcaaaggaatctttatttggctttggctcaaggctaggccttcatctcatcttctaaaacctagaaggaagctttcagaggaaaatatagatcagaagtaaaattcggattagaagttggggggctgaagttgtaggatacgagggagggaaagcctacctttacccttgaacaatcatcatgacaacaacctcctaagtcatcttgagtgtgcaaagtgcttttctgtacattgttcatttaaattgattcctcttcagtttagctagctaattatccaaaatgtctgtaaaaattatgacagaggagggaaacacagttactgatttggggtccattccctttacccttctcatattcaggtatttccccactgtgcagaattcactttgaatccagcggaaattacctaggaggaaagagctgggctcaggaaggaagcagacatttcactgtggctttattcacgggaggctctccagacgtttggcaggtcttttcttacatgtgcctactggaccttgtgttctaagaggcacagaacagctgtacacagcccagtgtcacacagcaagtcaggagcgagaatccatgtagaattaagatctttttttccctccctcctgaatctctatctagcccgtgtctactctcccacgggattttgtgttgctttttttaccgtctgtctcagcttcaaaaaggatctgttttctcttattattttcccacattgctgcggttcagaaccgtagtgcatttgtcaaatgcattgtgtgtgtcttcacccctgcagcttccctgtcagatgattcaatacaaggcaatgatctctccactaagggggaagaaagaaagtgagaaagaaagaaaacagctcagagcccttaaccctgggttgtggctatttttctcatagacaggctgctctctcttatctgattggagcctccgtgttctctgaataggaaataaatctctacacaaaaatccaagggttagtttccaggtgtttgataggaagctttcatcccaagtggtttgtttatttatttatttatttatttatttatttatttatttatttttgtcggggggagtttgttgcattatttgaccaaggacagtgttgaccacctccggtttctacttctctttcgaagtttattttatgttttgttgtggttttcagatttctcatggtttggatttgggaaagtaaaatcaagacaaggtgttggtaagtagttaactcactccttctttggataagtaatgagtctatgtttttattcggatgaaaatgcttagaccctctttacattccacggtctttgattcattttctgtgttcctggtttttgatttgttgaagaagtaggtctagaatttttccagaaaaggcattactgttgttaattgagtttagtacatggttttacatttttagatttgactcgtttttatcctgtgggggaagtgttaactgtcgttccagttctagtttgtgatgcatttttaagaaggttaaggaaataaatacaactacatttcagctacatgtacagggaatagctgactgaatagtcaagctggacaagtttaggtcttaactttcttccattggccaagatgtaatagagttctgaattggtaaatcagtttccatttatgaccattcctctttcaaaacagattctaacgtccagacgctgtgatttctgcttgattgcattctattccctgcattcagactatgtgctgacaattgaaaagcagtttgtagcttaccaagtgaatgcagatgatgtagctcctcattagaattgaaatcaggtgtgatctcttgggacctctaatttgttcagaattttctatgggtacagagacaatgaacaggaattgcttagaaggcatgaaaagacggcattcttacatctttcatgtgggtttccaagatccgaccatcttgggcatctgttggttagttattcttttggtagcaaggttggccataggtaaaatggcaaagtaggggaaatgtaccaatgtcaggaggctgctcaattccaaaccatccaccctatgcttgttcatcctggccatataaggagctgtttcctgtcactttccatcctgacgtcagaccttgtcttgttgggcagtctctatcactgtctgtccagtgatccaaaaatgaccagccggctgaggcagcctggccagatggggcctggaggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggctagaaaatttgtcttgtacaaaaaatgctatgattattaatttagtataaagacaccaagttatgactataaatagtattacagctaacaacaaagcaggtaactttgccatgggattgatccattattgttataaaagtattttgtagacaaaagtggtgcagatactcacagaatatgaagaaagaggaatgtagtcataggacagtcaaatgtatatctggtctaccagttaaagttcatttagtaatacctagtattaatacatactcatgaagcatatttaaatttataatacattttcctgttactaatctcgttctagtgtcataaaataagcaaatattattttgcatataagtcgatgtagaggtccaaagaaagtaagtgacttgtccaaagggtagagaaaggaaactcagatcctgaaattcccagaacaactttcaatgctctgtgaaatagggatcccagattcatgttgacagtttcaatgacagagtgttctagagacaatcatagaacagagaggaaagctacgtattatcttacaggagaaggaagtgaagaaattggagctctcatacactgctggtgggaatataaaatggtgcagccactttggaaaacagtttggcagttcctcaaaatgttaaatatagagctaccatatgacccagtaattccacccaagagaaattaaaacacgtgtccacataaaaattcgtatatgaatgttcatagtagcattattcataatagccaaaaagccagaacaacccaaatgtccaccaacagatgaatggataaataacatgcaacatatccacacaatggagtattattaggcaatacaaagaaaggaagtactaatacatactacaatgtggatgaatcttaaaaacatgatgttaagtgaaggaagccagttacaaaagaccacgtgttatatggttccattcatatgaaatgccagagagatagagtgtaaatctgtgaatgtctacggctgaaagtggagagaatggggagtgagtgctaactgggtgatgaaaatgtcctacaagtagattatagtgattattgtactactctgtgaatatactaaaacccactgaattgtatactttaaatgggtaaattttatgtatgtgaattacagctcaataaagccattgattacaggagaatatatatatttttccatctccaggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatgtaagtacagctgtttgtacttattgtcccatttgctgcatagctgtatcaaccagagtgtttatcagagcccctgatagggatgactggcggggaacagggtgcagtgaaatgtttgaggattcttgcagcttttggcccaggaatagatcctttagtttgttgcttatggaattctccgggtgtgtctcctgctgggaagggaactgtgggcagatactgactctcttgggatctattttccaaaaccatttgtcaatgtgaagttggaatcaaggcatgcttatcttgaaaaagagagacagaaaccagaagtgtcatttgaatgcacatatatcagttatagatgacaagcaatgaatgtaatttaaatttagcctatagaaagtaattgaaatatgcactctaggtagttgggaaggcactgtgttgcccacaggtggtatcagtttggggagtcctgtattctttgctcagtccgagttcccagcatgaggccttgttgtgttgccacaatagcaaagagacatggcagcagatgatttgcatattaagtacacttactttcatttttcaaaaacaaattaacccattaatctctgttagcaagcttgagatttttctaagggccttttgtgctatcttctgcaccactgctccaatattaaatgtgtagcacagccttaaagacaacaaaaaataaacccacagtggacaaacaggcttatggcattgcttccaggccttccttcatatgtgaataaggggccgtgagggctatgatgtcagatcccagagattccctgccactaaggagtgggtatcatgcatgttcctatcctgtcttaaataccattctcctaccttgtcatagaggtaaggtagaggattactgcaagattaggctttgggggctgcctctctgttgagagctttcatgggatgaagggccttagcaatgagtatcagatctgtgcatatataaggaaatatctgtaacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntacttgtaaaatgcagagaggattatgtcatgggattgtgagactgaaatgagagaatatagttggccacacattccaagctctagagggcatcacaatctctttttaatgcactaccattcagcaacaaggagttcagaaaataataccagccaagacccagcggaactcccgtgctaagggactcaacatttaacgtgctgagtgaggaggcacagctcacatgtacttctgtcctctgcagggtctccttgtgggcctgtcctccatgacctctgtcctttggtgtgctccctctctggtttccaccttgcagccaatgagcttctgaggacccttaactgcctcctgccttgtgcaccaagagggtgtatggatgaatgacttgagccgtcgctgctccagactgtagggaggaacattctcttgtcctgggattcatctgcttttgtctcttcttggccttggagagccaagactgagccatgattttaaaacacggctaaggaagctagtccttaatgcagacgtctagatgtttctgatccaaacaaaggaagtcagtgaggcccaaccatgttttagcccaccccactagaaggctaaaactgaggtgaggccaaaaccttaattaacaatgcaaagatcttgcattttccttttgtatcctctgactctgtctcccttaggaatctgagatggcttaaccaataattcaggattgctagctatctctctgctttgccctgctggacacactgctaacaaatatatgcaaagcatctggcttataagaagcctgccattaatgtgaatttctcatttctccccttttttactctataaagtttatcaaggatattttgcagtcagcaactaggtatctactttagttttatcagaaacaataactgagttctggagaaaaaggtgatgaaacctccgcccagccatttggggtggactgtgaagatggacacccagcttcttcggttcactgtgcacagaggagagactcttgggcctggaagctcaggtacaggccaccaactcttcacccgctgccttcacctccacatgcttcaattccaatttccattctgtctttgttttgcagttgttcagcacatcaagcgacataacattgttctgaaaagggagctaggcgaaggagcctttggaaaagtgttcctagctgaatgctataacctctgtcctgagcaggacaagatcttggtggcagtgaaggtaagagaacattccagaatgtctcattaaccatgatcacacttacgtgtggaaatttaactctattttattatattttggtggccaatgcaggagtaaattgttcctgctatgatggatgtattcaaattcctttaaactaaatggacagtggaaaggacatttggggcacatccaactgtcagaggttcttctcagtggccactgacaagtctgcaggctgtgaagagctctgagctactcttccaagtccatgctaactgaattttggatggaagtcagtggggccaaacagaattcgaccagctgagtccttcctgaagactcacatgtgtctcctcaaattgttaggtgcttgttccaagctcagctgaagcttgtgtcaggtttatggttctggaaggaaggattgtggacttcacttcccctctcagcctctgtgccactcaattctctaccctgaccctcccagatgcgtacaaatctccagggttttatatagcacaggcatgatttcatccagggctggatggcatctcctgtggaattcaccacccaaggagacatagtggttttactaaaatgaagactgaaagtcctccatcatagtcacgtcctcataaactcaaaaaggagtcctggtcttcaattagataatcattatgattgaaggggatacaatttccactgagctctctaaaacacaagaattaacaaaaatagcacaaaaggcttcaggtattttacatatatttttcaaaaaatttttaactcaccaaaataatgggtaatatatatttaaaacacttaaaaggggcatatatttataaaatgaattaagctccaaatgaatatggacatggctaatgtaataaaatgtaagttcttgacataatataagattattgagttttttgctgttttatttctgccaaacaggaaacatttttacaaatggcatttatttactagtcataaccaggacaacttttcattttagaaaatcaggagtctattccattactgttatacttaacattaaaatgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggacccttgtaatgacattgggccctcctggataatccaggataatctctccacctcaagatccttaacttagttacatctgcaaagctgcatttgccacatcaggcaacatattcacaggctcccaggattaggaagtgaacagccttggcaaaggtgaggagttcaaccttccatgccagtgaccacacccaatatagcattccaggcagaggaatgtgcacatgtgcaaagggagggaggcatggcctgttgtcatcggggcagctgcagacagttcctaacgtggatgcatgggtttcgagaggagtgtcaagggattgagctggagagagatgcaggtgctgattcattcaggacagtgggttttgagcttgaattgcagttatttatatacatgtcatatctccctatttgcccataagtgcctgaggccaggatctgggtcattgccttcttgatgtcatcacagctctggctcagtgatgggcacatgccaagagctcagcacattatttgcatcactaaatgccagctttgccctgttatgacaccacagtggcagagttatctaagcagagaactaaaggcaccaaaacccagctgtcatgctcctgggcatcttcaccttggcagcatgaccactcttcttgcggcttgctgggcagggatgttttgtgagccggcagtgtttgtgggcacaggctggagaaggctctgtgcagtgctacataaagacaggcaaactgatcgttgctgaagctggagatgggtccacaaaggttcactgtactcttctttctactgaatcataacataaagtttttaaatcttatctgagcagaggtaagctgcacatagcatcacagaaagaaagcacaacaggcaccaacaagggaaagaagcccgtgggaattattgggtgcagaccagggttagaaaggatgtggaccctaaaaagacctcctgtctgtaacctaatgagagaacctctgtgaaagtgtgtcataagctggaaagctttgcatatgcctaaggagttattcctcagttattagcacaaatgttatttccttaggaactaggctgttttctcatcttttgcctaacaaatgagatggatgtctttcctatctcagtatcatagggcccactgaagtaatccttctcttttaacacccatccccagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcaggtacagtgaggcggggaggtgggctccaggagggagcaggccttcagggttcaggagtggagggttcatgggagggaacaagggacccctggacctttctcgaagcatcttatttgataatgacaccagcatatgccagagaaaggaggagagaaaaaagaaggtgacagtcttgttctttttccttaatgaattctatgtaatgtgatgcaaatgatccaactcaattcagtccgcattcactgaacatttgctacatggagtggtatgtgacatgggcatttgggatgggttttagatgatctgtaggagtttgctatgtggtgttattaaaggcaaaggaaacaggaagagctaaggtacccaggtgtcacaatggaaattgcagtccagtgagagtgaagttttcaagttcccagactgtggaatgaacagtaaggaagggaagcagaaaaacagctggaggagtcaaggctgaaagggtggggcaggtgcttggactttatcctaaggacaaaaagaataaccaggaggccagtagttcagaggatctacagtggaaagatccaaatccaaagaagaaaagcttcagaaggaaggaggtaaaaacattgattgaagactccagattatgcggagaccctgaggtcatgaaaagatggacagcttcaactgagaagttttgtgggatgtggggtcttcagaaaaaggcaaggcaaggaagagaaaatgttgactataaagggagtcactgggctattgattagggttagtgtcatttgaatttctagcattggcctgattggttgggatttatttatttatttatttatttatttatttatttatttgagacagagtctcgctctgtcacccaggctggagtgcagttatgtggtcttggctcactgcaacctccgcctcccaggttcaagtgattctcccacctcaacctcccacgtagctgggataacaggcatgtgccaccatgtctggtaattttcatatttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncatttatgcagaatcctctatatttattctaagtgtttcaaaacgtcttgagagagatctttcctttcccagagagaagcccttggccacgccagggcagagcccgacagcaaaccccgcctttattccttgcactgcagcgccccctcaggccaccaggggcaatgacgctcatcttggctcacagcgcaggatgtcacagcagagcctgccgcgctgagggcggcatcctaagacgcggatcctcccacccttgcaggacttgcgtgttagggtgccctaagtcatcacatagctgaggagaggccaggtgtgtaagacagaagaaaaataagatttaaaaaatcatagtccccctccccatagtgtaaacaataaaattttccccagcaaaagaataaacaggctgtggttcaaaagagttctcctatgtgttggccatagcctgtgttgagcggaggttggaggaggggtggacagaaggaaatggtattttgtattgagaatgaacatccagattgagaatgccctgggactctgttgttgaactgggcttgagagagccaggcgtagcttcaaaaccatgcctggccacagatcaggtggactatggaaggccgcaggctgcccagctctggtgctggtggggacactctggaggaatcagcttggttgggggatactctggcatagcctggctaggtgggaaggggtggggacccactgggctgccatgtccatcccactaggagagtccatctttccccatatgttttgtttttggttttgtcttccttcacctacctgctgcttctcagggtcagggggccctggtaagtgcctgaacccagcacagtgccagagcccctaaaatgtcaacctgtctgtacactggagggaagggggacattcaggcccacggggtcctgggaattccagactctttgggaaacagagcagcctgggtgctctcaggactgcagaagtacaactgtgtgagcacctttgtctcctcttcatgctaagtcaggcagcatcttttagcaccagcagctacagggtgggggtgaggagcttagcaagagggacggggaggggcaggggcaaagggcccctggagtgaaaatgctgaggcccccagcttcattctccatgtccttccccagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtgagtagctgtgcagatcagagaccccagggacctctttccctgcgggacccctgctgtatttgtttgctgaggctgtcataacaaaatatcatgactgggtggcttaaacgacagacatgtgtttctcatggctctggaggctggaagtccaagctcaaggtgtgagtgggattggttcctcctgaggcctcgctccttggcttgcaggtggctgccttctcactatgccctcacattgggatggtgacaacccaatccttccctttaggcctaaatttcttcttttaaggacaccagtcagattggattgcatcccaccctaatggtctttttaacttaatcacctacttaaagtcttgtctccaaatacagtcacattttgaggccctggaggtgaggttatcaacgtataaattttggggggacgcaggtcagcccatagcacccaccctgctcttggcactccagcgtggtgggtgagatctccaagagtgaagtcctcagtgtgagaatccttccagagaaaactgagggcctgggggaattcctggtgaggttaaaggatgctgaggtttggctttgattctgtgaaagtatgcctggagcctttgtttgagatttgaacaaaagtgaacttgttcacataaataatagtacaccggactttggaagttccctccaagcgccatcttggttgaaagcggtttagcttggtgagatggaagaagttttatcctgcctgttcctagacacttccacacagcttgtctggtgtttccaccactgaaatcactgagaggcagagttacagacagtgactcagatccactgatgacctagaactggcccccgggatatgttaactttgttaactgagattttggaactcggacatgagagaaaagggaatttctggaaatgttgaagttgtgatatttagagtgaagattcggggtggtgttctaccactttgattttctaccaaccctcaatttgattttcttgtgtgttttgctgtttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntctttggtgctaagaacatcactgggaatcaccaagcatgttagacatctctgacatttcagcctcttgggttctggggtaggggaacattttccagacactattagtttattttcttcctttaataaatactaactggatattgagattagaattgggcagaaactctatgcaccattactctataaactcaggacccagagttgttagagaggtccaattttctgctgtaggataagcccacctgctctcagctgtcaggcatactgtgtagcatggtggtgctggggggttaccaggatgaaaagcagaacctaccgccacaggactactgtgcagaaatctgttcatgggtttgaaatcgggcatccctgacaaacctggaaatgagcttcctaactttcctccatgaagtaggaagtcatatacaattttactttgtcaaagaaagttttctttgcctgtactaatgcgattgctatctgatgccccagtctcagtgttttcattaaaatgggcatttaaaaaatgtatttatctattgtggtaaataatctaaggagcagtgccctgtaatagaggaaagtcctgagcttagaatccaaagacctgagttttgtttctgtaagcaatctgaatcttattgccttatttgtagaatgagaataatgatgcctgacctgcccatcgctgagtgatggtggaggggtttgaatcagcatcatgcacacataagacctgaaagcagaaggagactctggccttgccaaggaagtagagtcactggttggtcattttggatgcagagtacctctttgttctcctggtcaaagtgatcttgttaattagaagattgagtcatcctcttagcagaaagtgtgtgtatgtgtgtgtagttttatttatccaaggtgatcatctacctaatcatcatccctgctcctatctggttttgagagaaagaattgcagataatatttttttaaatgattttgatcactctttgccttctgtctctgttgcttgagactgtgaagaagtcatatatcctttatgtgtttataaacaattaaaactataaagtgcaaataaggaaagcaaacagtgtcccccagcagctcccttccacacctggtttcggggtgactgatgcctccctgttgatccctttctccccaggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgtgcaatgggtctggccaagaccctccagagggctgagatcccagaggcatccattggctggggccttgttttgggttggaagaccatgtcaggacacgatcttatgggctttgttggtggcagcacttcccaagtagcctgctatgtttctaaatttcatggtcaaagtataatcactctgagaaagcaaagcacagtgctcgctgctatgtccttgctcctctggtgatgggactgaggagtcactttatgcagctcatctgataaattaatagcagacgtagcattccagaatccatacagtccagcaagttctttagtattgttccaatgaagtttcaagattctttcctttttgaatatttcaccagaacattaacttctagtgaaaaactcaagcattttgttttttctattcttacgcttgcaaatattgtttatatttgaggcacagcatcttgatcaggcaccgtacatctttttcagatccatttctatctttttaatccagttcctttatccaatagcctgagaggtttcctgtcagtcagtgccttaacacccaggtgactcaggttccatctcagaccaataaaaaccacaacatgtgggagctccagtgtgaggtttgggaccattccatataaattatttttccaccagttttgtgaaatcagagtggcttggacaaatggatataagccaatcaaaataaacacgatataaactaacctataataattgctctcttccttctgagaaaacaaaaccaagagtgggcttcttttgtaaaagccctctcttctgctgtttttttgcactgacatttcttcgatgtgcattgcttttcctcctgtctcatcctatctttgatctccatccaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggctagggcccttttccccagaccgatccttcccaacgtactcctcagacgggctgagaggatgaacatcttttaactgccgctggaggccaccaagctgctctccttcactctgacagtattaacatcaaagactccgagaagctctcgagggaagcagtgtgtacttcttcatccatagacacagtattgacttctttttggcattatctctttctctctttccatctcccttggttgttcctttttctttttttaaattttctttttcttcttttttttcgtcttccctgcttcacgattcttaccctttcttttgaatcaatctggcttctgcattactattaactctgcatagacaaaggccttaacaaacgtaatttgttatatcagcagacactccagtttgcccaccacaactaacaatgccttgttgtattcctgcctttgatgtggatgaaaaaaagggaaaacaaatatttcacttaaactttgtcacttctgctgtacagatatcgagagtttctatggattcacttctatttatttattattattactgttcttattgtttttggatggcttaagcctgtgtataaaaaagaaaacttgtgttcaatctgtgaagcctttatctatgggagattaaaaccagagagaaagaagatttattatgaaccgcaatatgggaggaacaaagacaaccactgggatcagctggtgtcagtccctacttaggaaatactcagcaactgttagctgggaagaatgtattcggcaccttcccctgaggacctttctgaggagtaaaaagactactggcctctgtgccatggatgattcttttcccatcaccagaaatgatagcgtgcagtagagagcaaagatggcttccgtgagacacaagatggcgcatagtgtgctcggacacagttttgtcttcgtaggttgtgatgatagcactggtttgtttctcaagcgctatccacagaacctttgtcaactt

SEQ ID NO: 2 - DNA, 15 bp, Artificial Sequence
  ASO Probe for Detecting Alleles at PSs in Haplotypes Comprising Preferred Embodiments of Progression Markers I and Progression Markers II
gcctcccsgcacggg

SEQ ID NO: 3 - DNA, 15 bp, Artificial Sequence
  ASO Probe for Detecting Alleles at PSs in Haplotypes Comprising Preferred Embodiments of Progression Markers I and Progression Markers II
cttgcccytagggcc

SEQ ID NO: 4 - DNA, 15 bp, Artificial Sequence
  ASO Probe for Detecting Alleles at PSs in Haplotypes Comprising Preferred Embodiments of Progression Markers I and Progression Markers II
agacaagrgatgcag

SEQ ID NO: 5 - DNA, 15 bp, Artificial Sequence
  ASO Forward Primer for Detecting Alleles at PSs in Haplotypes Comprising Preferred Embodiments of Progression Markers I and Progression Markers II
gggaaggcctcccsg

SEQ ID NO: 6 - DNA, 15 bp, Artificial Sequence
  ASO Forward Primer for Detecting Alleles at PSs in Haplotypes Comprising Preferred Embodiments of Progression Markers I and Progression Markers II
tttctccttgcccyt

SEQ ID NO: 7 - DNA, 15 bp, Artificial Sequence
  ASO Forward Primer for Detecting Alleles at PSs in Haplotypes Comprising Preferred Embodiments of Progression Markers I and Progression Markers II
agtcaaagacaagrg

SEQ ID NO: 8 - DNA, 15 bp, Artificial Sequence
  ASO Reverse Primer for Detecting Alleles at PSs in Haplotypes Comprising Preferred Embodiments of Progression Markers I and Progression Markers II
cccccacccgtgcsg

SEQ ID NO: 9 - DNA, 15 bp, Artificial Sequence
  ASO Reverse Primer for Detecting Alleles at PSs in Haplotypes Comprising Preferred Embodiments of Progression Markers I and Progression Markers II
agctcgggccctarg

SEQ ID NO: 10 - DNA, 15 bp, Artificial Sequence
  ASO Reverse Primer for Detecting Alleles at PSs in Haplotypes Comprising Preferred Embodiments of Progression Markers I and Progression Markers II
cctgtcctgcatcyc

SEQ ID NO: 11 - DNA, 10 bp, Artificial Sequence
  Forward Primer Extension Oligos for Detecting Alleles at PSs in Haplotypes Comprising Preferred Embodiments of Progression Markers I and Progression Markers II
aaggcctccc

SEQ ID NO: 12 - DNA, 10 bp, Artificial Sequence
  Forward Primer Extension Oligos for Detecting Alleles at PSs in Haplotypes Comprising Preferred Embodiments of Progression Markers I and Progression Markers II
ctccttgccc

SEQ ID NO: 13 - DNA, 10 bp, Artificial Sequence
  Forward Primer Extension Oligos for Detecting Alleles at PSs in Haplotypes Comprising Preferred Embodiments of Progression Markers I and Progression Markers II
caaagacaag

SEQ ID NO: 14 - DNA, 10 bp, Artificial Sequence
  Reverse Primer Extension Oligos for Detecting Alleles at PSs in Haplotypes Comprising Preferred Embodiments of Progression Markers I and Progression Markers II
ccacccgtgc

SEQ ID NO: 15 - DNA, 10 bp, Artificial Sequence
  Reverse Primer Extension Oligos for Detecting Alleles at PSs in Haplotypes Comprising Preferred Embodiments of Progression Markers I and Progression Markers II
tcgggcccta

SEQ ID NO: 16 - DNA, 10 bp, Artificial Sequence
  Reverse Primer Extension Oligos for Detecting Alleles at PSs in Haplotypes Comprising Preferred Embodiments of Progression Markers I and Progression Markers II
gtcctgcatc

SEQ ID NO: 17 - DNA, 10 bp, Artificial Sequence
  10 base universal sequence
agcggataac

SEQ ID NO: 18 - DNA, 30 bp, Artificial Sequence
  Forward PCR NTRK2-specific Primer Sequences used in hME Assays
agcggataacatccaggacgacatccctag

SEQ ID NO: 19 - DNA, 30 bp, Artificial Sequence
  Forward PCR NTRK2-specific Primer Sequences used in hME Assays
agcggataacagttcctggagcttttcctc

SEQ ID NO: 20 - DNA, 30 bp, Artificial Sequence
  Forward PCR NTRK2-specific Primer Sequences used in hME Assays
agcggataacaactgacagatgaccctgtc

SEQ ID NO: 21 - DNA, 30 bp, Artificial Sequence
  Reverse PCR NTRK2-specific Primer Sequences used in hME Assays
agcggataacagttaagagagccgcaagcg

SEQ ID NO: 22 - DNA, 30 bp, Artificial Sequence
  Reverse PCR NTRK2-specific Primer Sequences used in hME Assays
agcggataaccattcacttccaggacctac

SEQ ID NO: 23 - DNA, 30 bp, Artificial Sequence
  Reverse PCR NTRK2-specific Primer Sequences used in hME Assays
agcggataactaggtcctggaagtgaatgc

SEQ ID NO: 24 - DNA, 17 bp, Artificial Sequence
  Extension Primers for Genotyping NTRK2 Polymorphic Sites
ctttcccccacccgtgc

SEQ ID NO: 25 - DNA, 19 bp, Artificial Sequence
  Extension Primers for Genotyping NTRK2 Polymorphic Sites
ctcctttttctccttgccc

SEQ ID NO: 26 - DNA, 19 bp, Artificial Sequence
  Extension Primers for Genotyping NTRK2 Polymorphic Sites
gatgaccctgtcctgcatc